(19) United States
(12) Patent Application Publication
(10) Pub. No.: US 2024/0307504 A1
(43) Pub. Date: Sep. 19, 2024

(54) INTRANASAL FORMULATIONS AND DELIVERY OF SOMATOSTATIN MIMETICS AND USES THEREOF

(71) Applicant: NBO PHARMA LLC, Bedford, NY (US)

(72) Inventors: Fredric D. PRICE, Bedford, NY (US); Barry R. FRANKEL, New York, NY (US); Dorian BEVEC, Bad Nauheim (DE); Warren WASIEWSKI, Lancaster, PA (US)

(73) Assignee: NBO PHARMA LLC, Bedford, NY (US)

(21) Appl. No.: 18/273,336

(22) PCT Filed: Jan. 20, 2022
(86) PCT No.: PCT/US2022/013144

§ 371 Date: Jul. 20, 2023

Related U.S. Application Data
(60) Provisional application No. 63/139,893, filed on Jan. 21, 2021.

(51) Int. Cl. A61K 38/31; A61K 9/00; A61K 9/16; A61K 47/10; A61K 47/12; A61K 47/26; A61K 47/38; A61P 25/00

(52) U.S. Cl. CPC A61K 38/31; A61K 9/0043; A61K 9/1611; A61K 9/1617; A61K 9/1623; A61K 47/10; A61K 47/12; A61K 47/26; A61K 47/38; A61P 25/00

(57) ABSTRACT

Methods and pharmaceutical compositions are provided for treating idiopathic intracranial hypertension (III) and cluster headache, wherein a somatostatin mimetic formulated for intranasal administration is administered, including direct nose-to-brain administration.

### BACKGROUND

Headache caused by idiopathic intracranial hypertension (IIH), cluster headaches and other related conditions are often debilitating and for which effective and easily administrable treatments are lacking. Moreover, such conditions can lead to permanent deficits; for example, the increased intracranial pressure (ICP) in IIH can damage the optic nerve resulting in permanent vision loss. At minimum, these conditions impact quality of life.

Systemic medications, life-style changes, and even surgical procedures are used to treat such conditions. Effective treatments that are non-invasive, easily compliant and facile to administer and needed.

Octreotide is a somatostatin mimetic (also referred to as a somatostatin receptor agonist) typically administered parenterally (subcutaneously or intravenously) for treatment of acromegaly; an oral form has recently been approved and intranasal forms have also been tested. Octreotide administered subcutaneously has been evaluated in treating IIH; however, repeated injections are undesirable. While oral dosing eliminates the pain and discomfort of injections, its timing requires careful attention to food intake; neither route is suitable for the patient population typically affected by IIH, cluster headaches and related conditions. Such conditions are therefore undertreated in the population. Moreover, numerous other conditions and diseases that are treatable by agonists of one of more somatostatin receptors lack easily compliant delivery means, thus afflicted patient populations are unaddressed.

A facile delivery route and formulation of a somatostatin mimetic amenable for safe and effective treatment of patients with IIH, cluster headaches or related conditions could reduce morbidity in the afflicted population.

### SUMMARY

In one aspect, the present disclosure provides methods for treating idiopathic intracranial hypertension (IIH) or cluster headache in a subject in need thereof, comprising administering to the subject an effective intranasal amount of a somatostatin mimetic formulated for intranasal administration. In some embodiment, the intranasal administration is direct nose-to-brain administration.

In some embodiments, the effective intranasal amount is less than the amount effective when the somatostatin mimetic is administered by a non-intranasal route in accordance with the same dosing regimen. In some embodiments, a serum area-under-the-curve from a unit dose of the intranasal amount of the somatostatin mimetic is less than the serum area-under-the-curve of an effective unit dose of the somatostatin mimetic administered by a non-intranasal route. In some embodiments, the non-intranasal route is subcutaneous, intramuscular, oral or intravenous.

In some embodiments, a serum area-under-the-curve from a unit dose of the intranasal amount of the somatostatin mimetic is less than the serum area-under-the-curve from an effective unit dose of the somatostatin mimetic administered by the intranasal route for an indication other than IIH or cluster headache. In some embodiments, the indication other than IIH or cluster headache is acromegaly or carcinoid syndrome.

In some embodiments, the effective intranasal amount of a somatostatin mimetic is about equal to or less than about 100 mcg daily. In some embodiment, the serum area-under-the-curve of the intranasal amount of the somatostatin mimetic is about equal to or less than about 50% of the serum area-under-the-curve of the somatostatin mimetic administered by a non-intranasal route. In some embodiments, the serum area-under-the-curve of the intranasal amount of the somatostatin mimetic is about equal to or less than about 25% of the serum area-under-the-curve of the somatostatin mimetic administered by a non-intranasal route. In some embodiments, the serum area-under-the-curve of the intranasal amount of the somatostatin mimetic is about equal to or less than about 10% of the serum area-under-the-curve of the somatostatin mimetic administered by a non-intranasal route. In some embodiments, the non-intranasal route is subcutaneous, intramuscular, oral or intravenous.

In some embodiments, the serum area-under-the-curve of the intranasal amount of the somatostatin mimetic is about equal to or less than about 50% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 25% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 10% of the serum area-under-the-curve of the somatostatin mimetic administered by the intranasal route for an indication other than IIH or cluster headache. In some embodiments, the indication other than IIH or cluster headache is acromegaly or carcinoid syndrome.

In some embodiments, the effective intranasal amount is administered as a single daily dose or as 2, 3 or 4 divided doses. In some embodiments, the effective intranasal amount is administered at a dose less frequently than daily, e.g., every other day, every third day, twice a week, or once weekly. In some embodiments, the effective intranasal amount is administered daily for a duration effective to treat or resolve the IIH or cluster headache. In some embodiments, treating is to reduce or prevent an acute, ongoing episode, and/or prophylactic and/or maintenance therapy to prevent future episodes. In some embodiments, the effective intranasal amount of somatostatin mimetic provides a minimal effective dose.

In some embodiments, the somatostatin mimetic is selected from somatostatin, octreotide, lanreotide, pasireotide, pentetreotide, or any combination thereof. In some embodiments, the somatostatin mimetic formulated for intranasal administration comprises a powder, liquid or gel.

In one aspect, the present disclosure provides a pharmaceutical composition in unit dose form comprising a somatostatin mimetic formulated for intranasal administration suitable for treatment of idiopathic intracranial hypertension (IIH) or cluster headache. In some embodiments, the intranasal administration is direct nose-to-brain administration.

In some embodiments, the unit dose comprising the somatostatin mimetic contains less somatostatin mimetic than would be effective if administered by a non-intranasal route following the same dosing regimen. In some embodiments, a serum area-under-the-curve of a unit dose of the somatostatin mimetic administered intranasally is less than the serum area-under-the-curve of the unit dose of the somatostatin mimetic administered by a non-intranasal route. In some embodiments, the non-intranasal route is subcutaneous, intramuscular oral or intravenous.

In some embodiments, a unit dose of the somatostatin mimetic formulated for intranasal administration is about equal to or less than about 100 mcg. In some embodiments, a unit dose of the somatostatin mimetic formulated for intranasal administration is about equal to or less than about 50 mcg. In some embodiments, a unit dose of the somatostatin mimetic formulated for intranasal administration is about equal to or less than about 10 mcg. In some embodiments, a unit dose of the somatostatin mimetic formulated for intranasal administration is about equal to or less than about 5 mcg. In some embodiments, a unit dose of the somatostatin mimetic formulated for intranasal administration is about equal to about 1 mcg.

In some embodiments, the serum area-under-the-curve of a unit dose of the somatostatin mimetic administered intranasally is about equal to or less than about 50% of the serum area-under-the-curve of a unit dose of the somatostatin mimetic administered by a non-intranasal route. In some embodiments, the serum area-under-the-curve of a unit dose of the somatostatin mimetic administered intranasally is about equal to or less than about 25% of the serum area-under-the-curve of a unit dose of the somatostatin mimetic administered by a non-intranasal route. In some embodiments, the serum area-under-the-curve of a unit dose of the somatostatin mimetic administered intranasally is about equal to or less than about 10% of the serum area-under-the-curve of a unit dose of the somatostatin mimetic administered by a non-intranasal route. In some embodiments, the non-intranasal route is subcutaneous, intramuscular or intravenous.

In some embodiments, a serum area-under-the-curve from a unit dose of the intranasal amount of the somatostatin mimetic is less than the serum area-under-the-curve from an effective single dose of the somatostatin mimetic administered by the intranasal route for an indication other than IIH or cluster headache. In some embodiments, the indication other than IIH or cluster headache is acromegaly or carcinoid syndrome.

In some embodiments, the unit dose is labeled for administration in a single daily dose or as 2, 3 or 4 divided doses. In some embodiments, the unit dose is labeled for administration at a dose less frequently than daily, e.g., every other day, every third day, twice weekly or once weekly. In some embodiments, the unit dose is labeled for daily administration for a duration effective to treat or resolve the IIH or cluster headache. In some embodiments, treatment is to reduce or prevent an acute, ongoing episode, and/or prophylactic and/or maintenance therapy to prevent future episodes. In some embodiments, the unit dose is a minimal effective dose.

In some embodiments, the somatostatin mimetic is selected from somatostatin, octreotide, lanreotide, pasireotide, pentetreotide, or any combination thereof. In some embodiments, the somatostatin mimetic formulated for intranasal administration comprises a powder, liquid or gel.

In some embodiments, A liquid pharmaceutical composition is provided comprising:
octreotide 0.1 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide Formulation 4—Octreotide 0.1 mg/mL, PEG 400 2% (w/v), polysorbate 80 2% (w/v), BAC 0.02% (w/v) in 0.01M citrate buffer pH 4.5

In some embodiment, the microcrystalline cellulose/sodium carboxymethycellulose comprises microcrystalline cellulose with about 11.3 to 18.8% sodium carboxymethylcellulose.

In some embodiments, a liquid pharmaceutical composition is provided comprising:
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

In some embodiments, a liquid pharmaceutical composition is provided consisting essentially of:
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

In some embodiments of the foregoing formulations, the compositions further comprise a tonicity adjusting agent such as mannitol, dextrose or sodium chloride, such that the tonicity is about 290 to about 500 mOsm/kg.

In some embodiments, use of the foregoing liquid pharmaceutical compositions is provided for nose-to-brain administration in a subject to treat idiopathic intracranial hypertension (IIH) or cluster headache. In some embodiments, the dose of octreotide is equal to or less than about 300 mcg per day, equal to or less than about 200 mcg per day, equal to or less than about 150 mcg per day, equal to or less than about 100 mcg per day, equal to or less than about 90 mcg per day, equal to or less than about 80 mcg per day, equal to or less than about 70 mcg per day, equal to or less than about 60 mcg per day, equal to or less than about 50 mcg per day, equal to or less than about 40 mcg per day, equal to or less than about 30 mcg per day, equal to or less than about 20 mcg per day, equal to or less than about 10 mcg per day, equal to or less than about 5 mcg per day. or about 1 mcg per day.

In some embodiments, a powder pharmaceutical composition is provided comprising
Octreotide 0.1%, mannitol 95%, hydrogenated lecithin 5%; or
Octreotide 0.5%, mannitol 95%, hydrogenated lecithin 5%; or
Octreotide 0.1%, calcium carbonate 95%, mannitol 2.5%, L-leucine 2.5%; or
Octreotide 0.5%, calcium carbonate 95%, mannitol 2.5%, L-leucine 2.5%.

In some embodiments, a powder pharmaceutical composition is provided consisting essentially of:
Octreotide 0.1%, mannitol 95%, hydrogenated lecithin 5%; or
Octreotide 0.5%, mannitol 95%, hydrogenated lecithin 5%; or
Octreotide 0.1%, calcium carbonate 95%, mannitol 2.5%, L-leucine 2.5%; or
Octreotide 0.5%, calcium carbonate 95%, mannitol 2.5%, L-leucine 2.5%.

In some embodiments of the foregoing, the powder pharmaceutical composition is prepared by spray drying, optionally at a temperature of 100° C. or 120° C.

In some embodiments, use of the foregoing powder pharmaceutical compositions is provided for nose-to-brain administration in a subject to treat idiopathic intracranial hypertension (IIH) or cluster headache. In some embodiments, the dose of octreotide is equal to or less than about 300 mcg per day, equal to or less than about 200 mcg per day, equal to or less than about 150 mcg per day, equal to or less than about 100 mcg per day, equal to or less than about 90 mcg per day, equal to or less than about 80 mcg per day, equal to or less than about 70 mcg per day, equal to or less than about 60 mcg per day, equal to or less than about 50 mcg per day, equal to or less than about 40 mcg per day, equal to or less than about 30 mcg per day, equal to or less than about 20 mcg per day, equal to or less than about 10 mcg per day, equal to or less than about 5 mcg per day. or about 1 mcg per day.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present disclosure and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

In one aspect, methods are provided for treating such conditions as idiopathic intracranial hypertension (IIH) and cluster headache by administering intranasally to a subject in need thereof an intranasal formulation of a somatostatin mimetic. In some embodiments, intranasal delivery is direct “nose-to-brain” delivery wherein the formulation is contacted with the olfactory area or region within the nasal cavity, enabling transport of compounds directly into the brain via olfactory neurons. In some embodiments, the dose and dosing regimen of the somatostatin mimetic provide relief from the symptoms such as headache and vision problems. In some embodiments wherein the condition or disease is acute or chronic, administration is acute or chronic, and may also be prophylactic to prevent the future occurrence of the condition or disease. These and other aspects of the methods, and pharmaceutical compositions for intranasal administration, are described in further detail below. In some embodiments, intranasal delivery is traditional intranasal delivery wherein a formulation is sprayed on or deposited on the respiratory area within the nasal cavity.

In other aspects, the intranasal delivery and formulations of somatostatin mimetics described here are useful for treatment of a number of other conditions and diseases in which an agonist or ligand of a somatostatin receptor (any one or more of somatostatin receptor subtypes 1-5) is salutary.

### Conditions and Diseases Treated

In some embodiments, a somatostatin mimetic may be used to treat acute or chronic idiopathic intracranial hypertension (IIH)—sometimes referred to as pseudotumor cerebri—, benign intracranial hypertension, primary or secondary IIH, cluster headache, pediatric intracranial hypertension, a pituitary tumor related headache, and related conditions, their symptoms and sequelae, by way of non-limiting examples. A subject may be treated as described herein for the first time after diagnosis of the condition or disease, or may be treated as described herein after failing one or more other therapies or one or more courses of one or more therapies. Furthermore, a subject may be treated to reduce or prevent an acute, ongoing episode, and/or treated prophylactically or with a maintenance dose therapy to prevent future episodes. Such rationales for starting and for continuation of dosing are full embraced herein.

As the nomenclature for these conditions and diseases has changed over time, some terms were or are synonyms for one or more others. This disclosure is not intended to exclude any such condition or disease because of changes in nomenclature or synonymy.

### Intracranial Hypertension

Intracranial Hypertension (IH) is characterized by increased pressure within the skull. Elevated cerebrospinal fluid (CSF) pressure presents with two symptoms, and can cause two problems: (1) headache, often severe; and (2) and visual changes. If the elevated CSF pressure remains untreated, permanent visual loss may occur.

Not only are there no medications approved for IIH, no medication for IIH has been evaluated in double-blind, placebo-controlled trials. Furthermore, no medication for IIH has been evaluated in ‘nose-to-brain’ intranasal delivery systems.

There are two categories of IH: primary intracranial hypertension and secondary intracranial hypertension. Primary intracranial hypertension, now known as idiopathic intracranial hypertension (IIH), occurs without known cause. This form is known to occur in young, overweight, females in their reproductive years (ages 20-45). However, IH can develop in both males and females of all ages and body types. Secondary intracranial hypertension has an identifiable cause including, but not limited, to drugs (such as tetracycline, lithium, vitamin A-derived oral acne medications or excessive ingestion of vitamin A, and oral or intrathecal steroids, growth hormone treatments, sleep apnea and certain systemic diseases such as lupus, leukemia, kidney failure (uremia), meningitis and dural venous sinus thrombosis. There is a known association of IH and Chiari type I malformation. In patients diagnosed with IH, it is critical to rule out an intracranial space occupying lesions by neuro-imaging (CT or MRI).

Although many factors are known to trigger the disease, the mechanism by which IH occurs, in either primary or secondary forms, is not known. In many cases, either type of IH may be chronic.

The most common symptom of any form of IH is often an unbearably painful or frequent headache, sometimes associated with nausea and vomiting, that is not relieved by any currently-available medication used off-label for this indication. The headache often awakens the patient from sleep. Some patients are treated in the emergency room where a lumbar puncture (spinal tap) is done to reduce ICP and to temporarily ease the headache. Measurement of the opening pressure is encouraged during these procedures in order to assess for intracranial hypertension.

The diagnosis is also confirmed by detecting a high spinal CSF pressure reading, usually greater than 250 mmH2O or 25 cmH2O (200-250 mmH2O or 20-25 cmH2O is considered borderline high) and normal laboratory and imaging studies including CT scan and/or MRI. There is generally a normal neurologic examination as well, although abnormal findings may be detected on eye examination. The eye findings may be subtle, and not noted in an emergency room evaluation. It is not uncommon to misdiagnose a patient with IH as simply having a refractory migraine headache, and be treated as such. Unlike primary IH, secondary IH patients may have abnormal scans and laboratory tests.

The high CSF pressure may cause the optic nerves to swell (papilledema). The optic nerve connects the interior of each eye, the retina, to the vision centers of the brain. The optic nerve transmits impulses from the retina to these brain centers. The earliest sign of papilledema on a visual field test is known as an enlarged blind spot. Abnormal CSF pressure can also affect the eye muscles controlling eye movements producing double vision, but this is an infrequent event. All patients with presumed IH should have a thorough eye examination including visual field tests by an ophthalmologist or neuro-ophthalmologist.

Other common symptoms include transient altered vision, particularly on movement or bending over, intracranial noise (pulse synchronous tinnitus), stiff neck, back and arm pain, pain behind the eye, exercise intolerance, and memory difficulties.

Affected Populations. The number of patients diagnosed with and afflicted by IIH varies by territory in the general population and the annual incidence ranges from about 1-3 per 100,000. For obese women of child-bearing age, the incidence is about 22.5/100,000 and the prevalence is about 85.7/100,000 IIH occurs in men and children as well, but at a lower frequency. Weight is not usually a factor in men and in children under 10 years of age.

The true incidence of secondary IH remains unknown because of the wide range of underlying causes and the lack of published surveys on the subject. Current statistics are not available on how many people have secondary intracranial hypertension.

Pediatric intracranial hypertension. This is the same disease; however, in the younger patients, obesity is not as common as it is in patients older than 14 years of age.

Related Disorders. Symptoms of the following disorders can be similar to those of IIH, and the pharmaceutical compositions and methods described here are also useful for their treatment.

Arachnoiditis is a progressive inflammatory disorder affecting the middle membrane surrounding the spinal cord and brain (arachnoid membrane). It may affect both the brain and the spinal cord and may be caused by foreign solutions (such as dye) being injected into the spine or arachnoid membrane. Symptoms may include severe headaches, vision disturbances, dizziness, nausea and/or vomiting. If the spine is involved, pain, unusual sensations, weakness and paralysis can develop.

Epiduritis is characterized by inflammation of the tough, outer canvas-like covering surrounding the brain and spinal cord known as the dura mater. Symptoms of this disorder can be similar to IIH.

Meningitis is an inflammation of the membranes around the brain and the spinal cord that can be caused by a number of different infectious agents such as bacteria, viruses, or fungi, or it may be caused by malignant tumors. Meningitis may develop suddenly or have a gradual onset. Symptoms may include fever, headache, a stiff neck, and vomiting. The patient may also be irritable, confused and go from drowsiness, to stupor to coma.

The signs and symptoms of intracranial hypertension and related conditions are not intended to be limiting in any way to the amenability of treatment following the guidance of this disclosure.

### Cluster Headache

Cluster headache. The term “cluster headache” refers to headaches that have a characteristic grouping of attacks. Cluster headaches can occur up to eight times per day during a cluster period, which may last 2 weeks to 3 months, or longer. The headaches may disappear completely (go into remission) for months or years, only to recur at a later date. A cluster headache typically awakens a person from sleep 1 to 2 hours after going to bed. These nocturnal attacks can be more severe than the daytime attacks.

Cluster headaches are an uncommon type of primary headaches (i.e., a headache that has no structural cause), affecting less than 1 in 1,000 people. Cluster headaches are a young person's disease, and the headaches typically start before age 30. Cluster headaches are more common in men, but more women are starting to be diagnosed with this disorder. The male-to-female gender headaches appear to be six times more common in men than women, especially men in their 20s or 30s.

No medication for cluster headaches has been evaluated in ‘nose-to-brain’ intranasal delivery systems.

The methods and pharmaceutical compositions disclosed herein are useful for the treatment of any of the aforementioned conditions and diseases, and those benefitted by agonizing one or more somatostatin receptor subtypes.

### Somatostatin and Somatostatin Mimetics

Somatostatin is a peptide hormone also known as growth hormone inhibiting hormone, that is produced by various cells in the body and has various physiological effects. Somatostatin mimetic as used herein refers to any natural form of somatostatin and any chemically related or unrelated molecule that has biological activity that mimics the biological activity of somatostatin. Somatostatin has a broad range of biological actions that include the regulation of neurotransmission and secretion and the inhibition of the release of growth hormone (GH), thyroid-stimulating hormone (TSH), gastrointestinal (GI) hormones, pancreatic enzymes and neuropeptides. It modulates the rate of gastric emptying, smooth muscle contraction, and intestinal blood flow. It also inhibits the proliferation of both normal and tumor cells. Two biological forms of somatostatin exist: somatostatin-14 and -28, which are derived from a 92-amino acid pro-somatostatin precursor. Somatostatin and its analogs bind to receptors belonging to the seven transmembrane G protein coupled receptor superfamily. Native somatostatin-14 binds to somatostatin receptor (SSTR) 1-4 with higher affinity, while somatostatin-28 is more SSTR5 selective.

The following provides a description of non-limiting examples of somatostatin mimetics useful for the purposed disclosed herein.

Somatostatin. The structure of somatostatin is shown below.

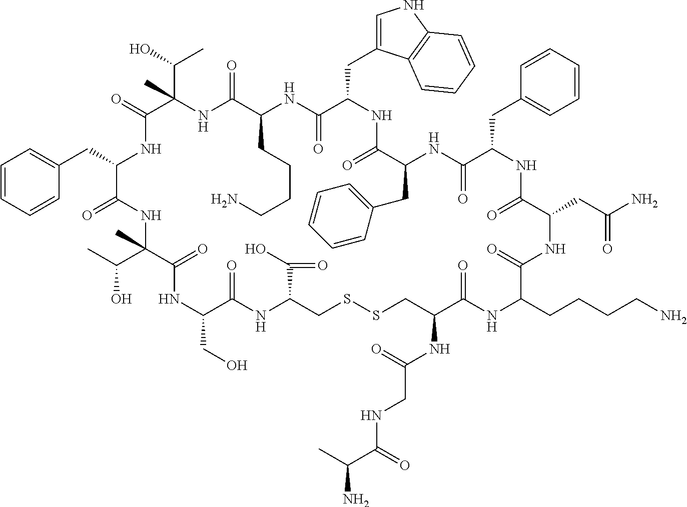 [SMILES: C[C@H](N)C(=O)NCC(=O)N[C@H]1CSSC[C@@H](C(=O)O)NC(=O)[C@H](CO)NC(=O)[C@](C)([C@@H](C)O)NC(=O)[C@H](Cc2ccccc2)NC(=O)[C@](C)([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)[C@H](CC(N)=O)NC(=O)C(CCCCN)NC1=O]

### Somatostatin

The IUPAC name is L-alanyl-glycyl-L-cysteinyl-L-lysyl-L-asparagyl-L-phenylalanyl-L-phenylalanyl-L-tryptophyl-L-lysyl-L-threonyl-L-phenylalanyl-L-threonyl-L-seryl-L-cysteine (3->14)-disulfide, and the IUPAC condensed nomenclature is H-Ala-Gly-Cys(1)-Lys-Asn-Phe-Phe-Trp-Lys-Thr-Phe-Thr-Ser-Cys(1)-OH, or in single-letter code, AGCKNFFWKTFTSC (wherein the C are disulfide linked). The molecular weight is 1637.9 g/mol. The chemical name is (4R,7S,10S,13S,16S,19S,22S,25S,28S,31S,34S,37R)-19,34-bis(4-aminobutyl)-31-(2-amino-2-oxoethyl)-37-[[2-[[(2S)-2-aminopropanoyl]amino]acetyl]amino]-13,25,28-tribenzyl-10,16-bis [(1R)-1-hydroxyethyl]-7-(hydroxymethyl)-22-(1H-indol-3-ylmethyl)-6,9,12,15,18,21,24,27,30,33,36-undecaoxo-1,2-dithia-5,8,11,14,17,20,23,26,29,32,35-undecazacyclooctatriacontane-4-carboxylic acid.

Somatostatin has two active forms produced by the alternative cleavage of a single preproprotein: one consisting of 14 amino acids (described above), the other consisting of 28 amino acids (H-Ser-Ala-Asn-Ser-Asn-Pro-Ala-Met-Ala-Pro-Arg-Glu-Arg-Lys-Ala-Gly-Cys-Lys-Asn-Phe-Phe-Trp-Lys-Thr-Phe-Thr-Ser-Cys-OH, disulfide bridge between Cys17 and Cys28). Having multiple synonyms including growth hormone-inhibiting hormone (GHIH), growth hormone release-inhibiting hormone (GHRIH), somatotropin release-inhibiting factor (SRIF), and somatotropin release-inhibiting hormone (SRIH), somatostatin is produced by delta cells of the digestive system, acts directly on the acid-producing parietal cells via a G-protein coupled receptor (which inhibits adenylate cyclase, thus effectively antagonizing the stimulatory effect of histamine) to reduce acid secretion. Somatostatin can also indirectly decrease stomach acid production by preventing the release of other hormones, including gastrin and histamine which effectively slows down the digestive process.

More relevant to the present disclosure, somatostatin is produced by neuroendocrine neurons of the ventromedial nucleus of the hypothalamus. These neurons project to the median eminence, where somatostatin is released from neurosecretory nerve endings into the hypothalamusial system through neuron axons. Somatostatin is then carried to the anterior pituitary gland, where it inhibits the secretion of growth hormone from somatotrope cells. The somatostatin neurons in the periventricular nucleus mediate negative feedback effects of growth hormone on its own release; the somatostatin neurons respond to high circulating concentrations of growth hormone and somatomedins by increasing the release of somatostatin, so reducing the rate of secretion of growth hormone. In the brain, somatostatin inhibits the release of growth hormone (GH), inhibits release of thyroid-stimulating hormone (TSH), inhibits adenyl cyclase in parietal cells, and inhibits release or prolactin (PRL).

Somatostatin (and its mimetics; non-limiting examples described elsewhere herein) are useful therapeutically to treat a number of conditions and diseases such as but not limited to acromegaly, pituitary and neuroendocrine tumors, VIPomas and carcinoid syndrome.

The biological half-life of somatostatin is 2-3 minutes, thus for therapeutic use, mimetics of somatostatin have been developed with longer biological half-lives. The disclosure herein embraces all such mimetics of somatostatin, whether modifications of the somatostatin peptide, or unrelated molecules with the biological activity of somatostatin.

Octreotide. Octreotide (SANDOSTATIN) is an octapeptide analogue of somatostatin with a biological half-life of 72 and 102 minutes after intravenous and subcutaneous administration, respectively. The chemical structure is shown below.

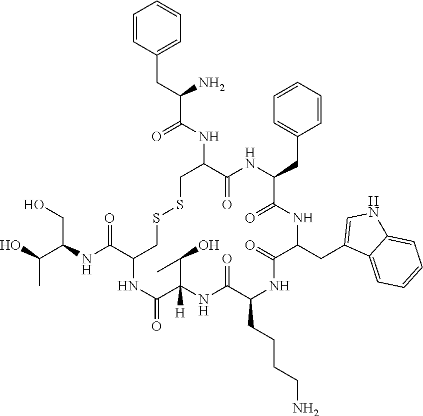 [SMILES: C[C@@H](O)[C@@H]1NC(=O)[C@H](CCCCN)NC(=O)C(Cc2c[nH]c3ccccc23)NC(=O)[C@H](Cc2ccccc2)NC(=O)C(NC(=O)[C@H](N)Cc2ccccc2)CSSCC(C(=O)N[C@H](CO)[C@@H](C)O)NC1=O]

### Octreotide

Octreotide mimics the biological activity of somatostatin, and is used for the treatment of growth hormone producing tumors (causing acromegaly and gigantism) when surgery is contraindicated, and treatment of pituitary tumors that secrete thyroid-stimulating hormone. Octreotide is marketed as an acetate salt.

Octreotide is available in a vial for injection (SANDOSTATIN Injection) in a buffered lactic acid solution for administration by deep subcutaneous (intrafat) or intravenous administration. It is available in 1 mL ampuls in three strengths: containing 50, 100 or 500 mcg octreotide (as acetate), and 5 mL multi-dose vials in two strengths, containing 200 and 1000 mcg/mL octreotide (as acetate). For treatment of acromegaly, dosage may be initiated at 50 mcg three times a day. The most common dose is 100 mcg three times a day but some patients required up to 500 mcg three times a day for maximum effectiveness. For carcinoid tumors, 100-600 mcg per day is suggested; in clinical studies the median daily maintenance dose was 450 mcg; some patients benefitted from as little as 50 mcg and some required up to 1500 mcg/day. For VIPomas, daily dosages of 200-300 mcg in 2-4 divided doses are recommended during the initial 2 weeks of therapy. For ongoing therapy, doses above 450 mcg/day are not usually required.

Octreotide is also available as an intramuscular (gluteal) formulation for depot administration (SANDOSTATIN LAR DEPOT). It is indicated for treatment in patients who have responded to and tolerated SANDOSTATIN Injection subcutaneous injection for: (1) Acromegaly, (2) Severe diarrhea/flushing episodes associated with metastatic carcinoid tumors and (3) Profuse watery diarrhea associated with Vasoactive Intestinal Peptide (VIP) secreting tumors (1.3). SANDOSTATIN LAR DEPOT is an injectable suspension in vials of 10 mg octreotide per 6 mL, 20 mg per 6 mL or 30 mg per 6 mL. The dosage for patients not currently receiving SANDOSTATIN injection subcutaneously is (1) Acromegaly: 50 mcg three times daily SANDOSTATIN Injection subcutaneously for 2 weeks followed by SANDOSTATIN LAR DEPOT 20 mg intragluteally every 4 weeks for 3 months; (2) Carcinoid Tumors and VIPomas: SANDOSTATIN Injection subcutaneously 100-600 mcg/day in 2-4 divided doses for 2 weeks followed by SANDOSTATIN LAR DEPOT 20 mg every 4 weeks for 2 months. For patients currently receiving SANDOSTATIN injection subcutaneously: (1) Acromegaly: 20 mg every 4 weeks for 3 months; (2) Carcinoid Tumors and VIPomas: 20 mg every 4 weeks for 2 months. For renal impairment, patients on dialysis: 10 mg every 4 weeks. For hepatic impairment, patients with cirrhosis: 10 mg every 4 weeks.

Octreotide is also available as a subcutaneous formulation for administration in a prefilled pen injector type device (BYNFEZIA) indicated for (1) reduction of growth hormone (GH) and insulin-like growth factor 1 (IGF-1) [somatomedin C] in adult patients with acromegaly who have had inadequate response to or cannot be treated with surgical resection, pituitary irradiation, and bromocriptine mesylate at maximally tolerated doses; (2) treatment of severe diarrhea/flushing episodes associated with metastatic carcinoid tumors in adult patients; and (3) treatment of profuse watery diarrhea associated with vasoactive intestinal peptide tumors (VIPomas) in adult patients. The device contains 2.8 mL of 2,500 mcg/mL octreotide. The doses are: (1) acromegaly: initiate dosage at 50 mcg three times daily. Typical dosage is 100 mcg three times a day; (2) carcinoid Tumors: 100-600 mcg daily in 2-4 divided doses for first 2 weeks; (3) VIPomas: 200-300 mcg daily in 2-4 divided doses for first 2 weeks.

Octreotide (acetate) is also available in a delayed-release capsule for oral use called MYCAPSSA. For efficacy, administration of oral octreotide must not occur in proximity of a meal (e.g., at least one hour before or two hours after). Such dose timing restriction may not coincide with the need for treatment or a patient's schedule, for those indications described herein.

Lanreotide. Lanreotide (SOMATULINE DEPOT) is a synthetic polypeptide analogue of somatostatin that resembles the native hormone in its ability to suppress levels and activity of growth hormone, insulin, glucagon and many other gastrointestinal peptides.

Lanreotide acetate is a synthetic cyclical octapeptide analog of the natural hormone, somatostatin. Lanreotide acetate is chemically known as [cyclo S-S]-3-(2-naphthyl)-D-alanyl-L-cysteinyl -L-tyrosyl-D-tryptophyl-L-lysyl-L-valyl-L-cysteinyl-L-threoninamide, acetate salt. Its molecular weight is 1096.34 (base) and its amino acid sequence is:

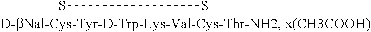

where x=1.0 to 2.0. Because its half-life is longer than somatostatin, lanreotide can be used clinically to treat neuroendocrine tumors that secrete excessive amounts of growth hormone (acromegaly) or other active hormones or neuropeptides. Lanreotide has many side effects including suppression of gall bladder contractility and bile production, and maintenance therapy may cause cholelithiasis and pancreatitis as well accompanying liver injury. SOMATULINE DEPOT is a somatostatin analog indicated for: (1) the long-term treatment of acromegalic patients who have had an inadequate response to or cannot be treated with surgery and/or radiotherapy; (2) the treatment of adult patients with unresectable, well-or moderately-differentiated, locally advanced or metastatic gastroenteropancreatic neuroendocrine tumors (GEP-NETs) to improve progression-free survival; (3) the treatment of adults with carcinoid syndrome; when used, it reduces the frequency of short-acting somatostatin analog rescue therapy. It is for deep subcutaneous injection only, in the superior external quadrant of the buttock. Recommended dosages are: (1) Acromegaly: 90 mg every 4 weeks for 3 months. Adjust thereafter based on GH and/or IGF-1 levels; (2) GEP-NETs: 120 mg every 4 weeks; (3) carcinoid Syndrome: 120 mg every 4 weeks.

Pasireotide. Pasireotide (SIGNIFOR and SIGNIFOR LAR) is a synthetic polypeptide analogue of somatostatin that resembles the native hormone in its ability to suppress levels and activity of growth hormone, insulin, glucagon and many other gastrointestinal peptides. The structure is:

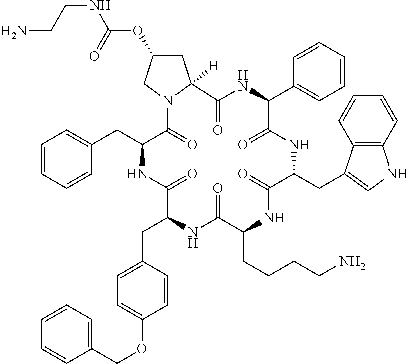 [SMILES: NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O]

### Pasireotide

Because its half-life is longer than somatostatin, pasireotide can be used clinically to treat neuroendocrine pituitary tumors that secrete excessive amounts of growth hormone causing acromegaly, or adrenocorticotropic hormone (ACTH) causing Cushing disease. Pasireotide has many side effects including suppression of gall bladder contractility and bile production, and maintenance therapy can cause cholelithiasis and accompanying elevations in serum enzymes and bilirubin.

The foregoing descriptions of various somatostatin mimetics is intended to be illustrative and non-limiting with regard to the somatostatin mimetics useful for the purposes described herein.

### Intranasal Formulations and Delivery Devices

Any of the foregoing, non-limiting examples of a somatostatin analogue may be used in an intranasal formulation and delivery system for the treatment of conditions and diseases such as and including those described herein. In one aspect, intranasal delivery is via direct (also called deep) nose-to-brain delivery, e.g. to the olfactory region of the nasal cavity, in one embodiment, to directly access the blood brain barrier.

Direct “nose-to-brain” delivery of a somatostatin mimetic may be achieved by use of any one of several methods that comprise a nasal formulation and/or a nasal delivery device to deliver the somatostatin mimetic to the roof of the nasal cavity, where transport into the central nervous system (CNS) is achieved. Nose-to-brain delivery is a minimally invasive drug administration pathway, which bypasses the blood-brain barrier as the drug is directed from the nasal cavity to the brain.

As will be seen in the description below, the efficacious dose of a somatostatin mimetic for the conditions and diseases described herein and in general those benefitted by an intranasally delivered somatostatin mimetic, is substantially lower than the dose required by a non-intranasal route. Moreover, the efficacious dose of a somatostatin mimetic for the conditions and diseases described herein and in general those benefitted by the direct nose-to-brain delivery of a somatostatin mimetic, is substantially lower than the dose required by a non-intranasal route. Moreover, the efficacious dose of a somatostatin mimetic for the conditions and diseases described herein and in general those benefitted by the direct nose-to-brain delivery of a somatostatin mimetic, is substantially lower than the dose required by a nasal route of administration that is not nose-to-brain administration.

A somatostatin mimetic may be formulated in, and administered by, a formulation and/or nasal delivery device wherein the somatostatin mimetic is delivered into the nasal cavity, e.g., to the olfactory region of the nasal cavity, enters the brain circulation and treats a condition or disease such as and including those described herein. Intranasal formulations and delivery devices provide such administration, and direct (or deep) nose-to-brain delivery further enhances the administration and further reduces the efficacious dose. See, for example, Wang et al., 2019, “Nose-to-Brain Delivery, Journal of Pharmacology and Experimental Therapeutics September 2019, 370 (3) 593-601. As disclosed herein, achieving delivery of a somatostatin mimetic into the brain circulation may comprise the use of uptake enhancers and other components, to achieve absorption via the nose-to-brain route using powder, liquid or gel formulations. The disclosure herein is not limited to any particular method or formulation for achieving delivery of the somatostatin mimetic intranasally, e.g., nose-to-brain delivery. As will be described further below, in some embodiments, the amount of an intranasally, e.g., nose-to-brain, delivered somatostatin mimetic necessary to achieve the treatment of a condition of disease described herein is substantially reduced compared to the amount of the somatostatin mimetic needed for efficacious treatment delivered by a non-intranasal route such as intravenous, intramuscular, oral or subcutaneous, or substantially reduced compared to an intranasal delivery method that is not directed to nose-to-brain. In some aspects, the efficacious amount of somatostatin mimetic for treating IIH and cluster headache is substantially reduced compared with the amount needed to treat a different condition or disease, such as acromegaly, by the intranasal route. While not wishing to be bound by theory, treatment of IIH and cluster headache using a somatostatin mimetic delivered into the brain by the intranasal route may be achieved with a substantially lower amount of somatostatin mimetic than heretofore administered by other routes for treatment of the other conditions and diseases described herein, and a substantially lower amount than heretofore administered intranasally for treatment of conditions and disease not among those described herein, such as targets outside of the central nervous system. As will be described below, the dose (amount per nasal administration) and dosing regimen (e.g., frequency of intranasal administration of the dose per day, duration of dosing in days or longer, among others) factor into achieving an efficacious brain exposure of a somatostatin mimetic delivered intranasally for the treatment of IIH, cluster headaches, among other conditions and diseases.

Examples of formulations useful for achieving nose-to-brain delivery of a somatostatin mimetic are described herein, but are not intended to be limiting. Non-limiting examples of agents useful for preparing formulations herein include bioadhesives polymers such as those described by Illum et al., The nasal delivery of peptides and proteins, T Biotech, 9, 284-289, 1991, incorporated herein by reference. Non-limiting examples of bioadhesives polymers include mucoadhesive polymers. Non-limiting examples of mucoadhesive polymers include chitosan, amylose, amylopectin, carbopol, cellulose, carboxymethylcellulose, sodium alginate, gellan gum, hyaluronan and poloxamer. Such polymers may be used singly or in any combination in a formulation disclosed herein. Use of chitosan is described in Ritthdej, 2011, Nasal delivery of peptides and proteins with chitosan and related mucoadhesive polymers, Peptide and Protein Delivery, Chapter 3, 47-68, incorporated herein by reference.

The present disclosure provides for somatostatin mimetic formulations that comprise one or more agents that facilitate or enable nose-to-brain delivery. Such agents are provided to overcome barriers to absorption include those that prevent degradation, enhance barrier permeability by transient opening of tight junctions, disrupting lipid bilayer packing/complexation/carrier/ion pairing and enhancing resident time/slowing down Mucociliary activity, as non-limiting examples.

In one embodiment, somatostatin mimetic formulations disclosed herein comprise one or more permeation enhancers. By way of non-limiting examples, such compounds are classified as surfactants, cyclodextrins, protease inhibitors, cationic polymers and tight junction modulators may be formulated with a somatostatin mimetic to facilitate nose-to-brain delivery. One or more of such types of compounds may be included in a formulation as disclosed herein. It is recognized that both specific and general formulation components and agents described herein may fall into more than one type of category; thus, one or more of any such compounds regardless of categorization may be a component of a formulation herein.

Surfactants. Surface active agents, or surfactants, are amphiphilic molecules possessing both lipophilic and hydrophilic residues. Surfactants have various applications in nasal drug administration, due to their high interfacial activity; one of which is as an absorption enhancer. Surfactants can enhance absorption with more than one mechanism; these include perturbing the cell membrane by leaching of membrane proteins, opening of tight junctions, or preventing enzymatic degradation of the drugs. Surfactants used as absorption enhancers can be classified as phospholipids, bile salts, (such as sodium taurocholate etc.), non-ionic surfactants (e.g. sorbitan ester such as monolaurate and monostearate, polysorbates such as polysorbate 20 and polysorbate 80), salt of fatty acids and alkyl glycosides (e.g., tetradecylmaltoside, N-lauryl-β-D-maltopyranoside etc.).

Cyclodextrins. Cyclodextrins are a family of cyclic oligosaccharides consisting of a macrocyclic ring of glucose subunits joined by α-1,4 glycosidic bonds. Cyclodextrins are composed of 5 or more α-D-glucopyranoside units linked 1->4, as in amylose (a fragment of starch). Typical cyclodextrins contain a number of glucose monomers ranging from six to eight units in a ring, creating a cone shape: α (alpha)-cyclodextrin: 6 glucose subunits, β (beta)-cyclodextrin: 7 glucose subunits, γ (gamma)-cyclodextrin: 8 glucose subunits. The inclusion compounds of cyclodextrins with hydrophobic molecules are able to penetrate body tissues including mucosal penetration of drugs. A cyclodextrin may be included in a formulation disclosed herein.

In some embodiments, a permeation enhancer used in a formulation as disclosed herein is a calcium-binding agent.

Cationic polymers. Polymeric systems with positive charges or modified with cationic entities, incorporated on their backbone and/or side chains, are called cationic polymers. These polymers are able to enhance absorption of macromolecules. Cationated gelatins, cationated pullulans, poly-L-arginine, polyethyleneimine, chitosan and its derivatives, an derivatives of any of the foregoing, are non-limiting examples of cationic polymers useful for the purposes disclosed herein. Cationic polymers interact with the mucosal barriers and enhance the absorption of water-soluble macromolecules via tight junction modification. Chitosan is a bioadhesive, cationic polysaccharide that are able to transiently modulate the paracellular permeability of intestinal and nasal epithelia enhancing the absorption of small and macromolecular compounds. Other tight junction modulators effective on tight junction proteins include claudins (e.g., CLDN1, CLDN2, CLDN3, CLDN4, CLDN5, CLDN6, CLDN7, CLDN8, CLDN9, CLDN10, CLDN11, CLDN12, CLDN13, CLDN14, CLDN15, CLDN16, CLDN17, CLDN18, CLDN19, CLDN20, CLDN21, CLDN22, CLDN23) and zonnula occludans (ZO) proteins (e.g., ZO-1, ZO-2, ZO-3).

Mucoadhesives. As described above, non-limiting examples of mucoadhesive polymers include chitosan, amylose, amylopectin, carbopol, cellulose, dextran, carboxymethylcellulose, sodium alginate, gellan gum, hyaluronan and poloxamer. Such polymers may be used singly or in any combination in a formulation disclosed herein.

Buffers. Non-limiting examples of aqueous buffers useful in a somatostatin mimetic formulation disclosed herein include citrate, TRIS, HEPES, acetate, phosphate, phosphate-buffered saline, MOPS and MES. A formulation disclosed herein may comprise one or more buffering agents.

Preservatives. Formulations described herein may further comprise one or more preservatives to inhibit microbial activity and maintain stability of the somatostatin mimetic, including anti-oxidants. Non-limiting examples of preservatives useful herein include benzalkonium chloride (BAC), thimerosal, benzyl alcohol, butylated hydroxytoluene, butylated hydroxyanisole, chlorobutanol, EDTA, menthol, methylparaben, propylparaben, potassium sorbate,

Viscosity modifiers. Non-limiting examples of viscosity modifiers useful in a somatostatin mimetic formulation disclosed herein include hydroxypropylmethyl cellulose (hypromellose; HPMC), microcrystalline cellulose (MCC), MCC/carboxymethylcellulose mixtures, dextran and chitosan, and any combination thereof.

Tonicity modifiers. Non-limiting examples of tonicity modifiers useful in a somatostatin mimetic formulation disclosed herein include mannitol, dextrose, sodium chloride, sorbitol and maltitol, and any combination thereof.

Other component may be included in formulations disclosed herein, such as a aerosol flowability enhancer or dispersant, e.g., silicon dioxide, L-leucine, dileucine (Leu-Leu), trileucine (Leu-Leu-Leu), Leu-His-Leu, and Lys-Gly-Asp-Ser, by way of non-limiting examples.

Non-limiting examples of other components in a formulation disclosed herein include lactose, water-absorbing, water-insoluble, water-swellable or water-soluble polymers such as polyacrylates such as sodium polyacrylate, potassium polyacrylate and ammonium polyacrylates; lower alkyl ethers of cellulose such as methyl cellulose, hydroxyethyl cellulose, hydroxypropyl cellulose and sodium carboxymethyl cellulose; polyvinyl pyrrolidone, amylose, polyethylene glycol e.g. of MW from 400 to 8000, hydroxypropylmethyl cellulose, microcrystalline cellulose, cellulose, α-cellulose, and cross-linked sodium carboxymethyl cellulose, water-absorbing and water-insoluble starches such as hydroxypropyl starch, carboxymethyl starch, water-absorbing and water-insoluble proteins such as gelatin, casein; water-absorbing and water-insoluble gums such as gum arabic, tragacanth gum and glucomannan; and cross-linked vinyl polymers such as cross-linked polyvinyl pyrrolidone, cross-linked carboxyvinyl polymer or its salt, cross-linked polyvinyl alcohol and polyhydroxyethylmethylacrylate. Solvents including water, ethyl alcohol, propylene glycol, etc. may be used in preparing formulations including those to be spray dried or otherwise prepared as powder formulations.

Thus, in some embodiments, a liquid or powder formulation for nose-to-brain delivery of a somatostatin mimetic such as but not limited to octreotide may comprise any one of more of the following components: a permeation enhancer, and/or a mucoadhesive, and/or a protease inhibitor, and/or a buffer, and/or a preservative.

For powder formulations, such formulation may comprise any one or more of the foregoing components, which are typically lyophilized, spray dried, agglomerated, milled, any combination thereof, or otherwise formed into a powder from a liquid formulation. Such powder formulations may comprise a carrier such as but not limited to calcium carbonate, mannitol, lecithin, lactose, sorbitol, maltitol, or any combination thereof.

For gel formulations, such formulations may comprise thixotropic components such as but not limited to hypromellose, pectin, gellan gum, and sodium hyaluronate. Such gelling agents are provided to remain as a gel at rest but produce fine droplets on shear thinning when a pump is actuated. Gel formulations comprising octreotide for nose-to-brain delivery are embraced herein.

The aforementioned components may be provided in liquid, powder or gel formulations for achieving nose-to-brain delivery of a somatostatin mimetic. Typically, the formulation is used with a delivery device to deliver the formulation into the nose, e.g., into or onto the olfactory region in the nasal cavity. Non-limiting examples of such devices are described herein below. In some instances, specific devices are used for specific formulations, as specific devices or formulations were developed based on using specific formulations or devices, but the requirement for a specific device is not so limiting provided it delivery the desired formulation and achieves effective nose-to-brain delivery. The ensuing descriptions provide exemplary liquid and powder formulations as guidance in achieving the purposes disclosed herein.

For any of the formulations disclosed herein, the amount or unit dose of somatostatin mimetic e.g. octreotide, delivered per administration may be from less than about 500 mcg to about 1 mcg. In some formulations, the amount of somatostatin mimetic, e.g., octreotide, is 300 mcg per unit dose, equal to or less than about 250 mcg per unit dose, equal to or less than about 200 mcg per unit dose, equal to or less than about 100 mcg per unit dose, equal to or less than about 90 mcg per unit dose, equal to or less than about 80 mcg per unit dose, equal to or less than about 70 mcg per unit dose, equal to or less than about 60 mcg per unit dose, equal to or less than about 50 mcg per unit dose, equal to or less than about 40 mcg per unit dose, equal to or less than about 30 mcg per unit dose, equal to or less than about 20 mcg per unit dose, equal to or less than about 10 mcg per unit dose, equal to or less than about 5 mcg per unit dose, or equal to or greater than about 1 mcg per unit dose.

Thus, for example, for a delivered dose of 10 micrograms of octreotide using a device that delivers 10 mg per pump, the concentration of octreotide in the formulation would be 0.1%. For a delivered dose of 10 micrograms of octreotide using a device that delivers 50 mg per pump, the concentration of octreotide in the formulation would be 0.02%. For a delivered dose of 80 micrograms of octreotide using a device that delivers 80 mg per pump, the concentration of octreotide in the formulation would be 0.1%. For a delivered dose of 80 micrograms of octreotide using a device that delivers 50 mg per pump, the concentration of octreotide in the formulation would be 0.16%. For a delivered dose of 50 micrograms of octreotide using a device that delivers 10 mg per pump, the concentration of octreotide in the formulation would be 0.5%. For a delivered dose of 100 micrograms of octreotide using a device that delivers 20 mg per pump, the concentration of octreotide in the formulation would be 0.5%.

### Liquid Formulations

Excipients useful for preparing liquid formulations disclosed herein may include but are not limited to polyethylene glycol 400 (PEG400), mannitol, Avicel (e.g., Avicel CL611), lecithin and polysorbate 80 (TWEEN 80), in addition to those described elsewhere herein. Such excipients may be included singly or in any combination. Non-limiting examples of liquid formulations suitable for the purposes described herein include those set forth below. Abbreviations: BAC, benzalkonium chloride; PEG, polyethylene glycol. Percentages of octreotide are shown as free base.

In some embodiments, a liquid formulation comprises about 0.1 to about 1 mg/mL octreotide; about 0.5 to about 10% of a polymer such chitosan, amylose, amylopectin, carbopol, cellulose, carboxymethylcellulose, sodium alginate, gellan gum, hyaluronan or poloxamer, or any combination thereof; about 0.5 to about 10% of a surfactant such as a bile salt, phospholipid, alkylglycoside, polysorbate 80; and about 0.005 to about 0.1% of a preservative. In some embodiments, the formulation is provided in a buffer such as citrate, TRIS, HEPES, acetate, phosphate, phosphate-buffered saline, MOPS or MES. In some embodiments the PH is about 4-6.

In some embodiments, a liquid formulation comprises about 0.1 to about 1 mg/mL octreotide, about 0.5 to about 10% (v/v) microcrystalline cellulose/sodium carboxymethycellulose (e.g., Avicel CL611), about 0.5 to about 10% polysorbate 80 (w/v), and about 0.005 to about 0.1% BAC (w/v). In some embodiments, the formulation is provided in a citrate buffer, e.g., about 0.025 M to about 0.05 M citrate, and a pH of about 4-6.

In some embodiments, the liquid formulation consists essentially of about 0.1 to about 1 mg/mL octreotide, about 0.5 to about 10% (v/v) microcrystalline cellulose/sodium carboxymethylcellulose (e.g., Avicel CL611), about 0.5 to about 10% polysorbate 80 (w/v) , and about 0.005 to about 0.1% BAC (w/v). In some embodiments, the formulation further consists of a citrate buffer, e.g., about 0.025 M to about 0.05 M, and a pH of about 4-6.

In some embodiments, the formulation comprises octreotide 0.1 mg/mL, Avicel CL611 2% (w/v), polysorbate 80 2% (w/v), BAC 0.02% (w/v) in 0.01M citrate buffer pH 4.5. In some embodiments the formulation comprises octreotide 1.0 mg/mL, Avicel CL611 2% (w/v), polysorbate 80 2% (w/v), BAC 0.02% (w/v) in 0.01M citrate buffer pH 4.5.

In some embodiments, the formulation consists essentially of octreotide 0.1 mg/mL, Avicel CL611 2% (w/v), polysorbate 80 2% (w/v), BAC 0.02% (w/v) in 0.01M citrate buffer pH 4.5. In some embodiments the formulation consists essentially of octreotide 1.0 mg/mL, Avicel CL611 2% (w/v), polysorbate 80 2% (w/v), BAC 0.02% (w/v) in 0.01M citrate buffer pH 4.5.

In some embodiments, the octreotide formulation comprises Avicel CL611 2% (w/v), polysorbate 80 2% (w/v), BAC 0.02% (w/v) in 0.01M citrate buffer pH 4.5. In some embodiments the formulation comprises octreotide 1.0 mg/mL, Avicel CL611 2% (w/v), polysorbate 80 2% (w/v), BAC 0.02% (w/v) in 0.01M citrate buffer pH 4.5.

In some embodiments, the octreotide formulation consists essentially of octreotide and Avicel CL611 2% (w/v), polysorbate 80 2% (w/v), BAC 0.02% (w/v) in 0.01M citrate buffer pH 4.5. In some embodiments the formulation consists essentially of octreotide 1.0 mg/mL, Avicel CL611 2% (w/v), polysorbate 80 2% (w/v), BAC 0.02% (w/v) in 0.01M citrate buffer pH 4.5.

In some embodiments, the liquid formulation comprises about 0.1 to about 1 mg/mL octreotide, about 0.5 to about 10% (v/v) polyethylene glycol 400, about 0.5 to about 10% polysorbate 80 (w/v), and about 0.005 to about 0.1% BAC (w/v). In some embodiments, the formulation is provided in a citrate buffer, e.g., about 0.025 M to about 0.05 M, and a pH of about 4-6.

In some embodiments, the liquid formulation consists essentially of about 0.1 to about 1 mg/mL octreotide, about 0.5 to about 10% (v/v) polyethylene glycol 400, about 0.5 to about 10% polysorbate 80 (w/v) , and about 0.005 to about 0.1% BAC (w/v). In some embodiments, the formulation further consists of a citrate buffer, e.g., about 0.025 M to about 0.05 M, and a pH of about 4-6.

In some embodiments, the formulation comprises octreotide 1.0 mg/mL, PEG 400 2% (w/v), polysorbate 80 2% (w/v), BAC 0.02% (w/v) in 0.01M citrate buffer pH 4.5. In some embodiments the formulation comprises octreotide 0.1 mg/mL, PEG 400 2% (w/v), polysorbate 80 2% (w/v), BAC 0.02% (w/v) in 0.01M citrate buffer pH 4.5.

In some embodiments, the formulation consists essentially of octreotide 1.0 mg/mL, PEG 400 2% (w/v), polysorbate 80 2% (w/v), BAC 0.02% (w/v) in 0.01M citrate buffer pH 4.5. In some embodiments the formulation consists essentially of octreotide 0.1 mg/mL, PEG 400 2% (w/v), polysorbate 80 2% (w/v), BAC 0.02% (w/v) in 0.01M citrate buffer pH 4.5.

In some embodiments, the octreotide formulation comprises PEG 400 2% (w/v), polysorbate 80 2% (w/v), BAC 0.02% (w/v) in 0.01M citrate buffer pH 4.5. In some embodiments the formulation comprises octreotide 0.1 mg/mL, PEG 400 2% (w/v), polysorbate 80 2% (w/v), BAC 0.02% (w/v) in 0.01M citrate buffer pH 4.5.

In some embodiments, the formulation consists essentially of octreotide and PEG 400 2% (w/v), polysorbate 80 2% (w/v), BAC 0.02% (w/v) in 0.01M citrate buffer pH 4.5. In some embodiments the formulation consists essentially of octreotide 0.1 mg/mL, PEG 400 2% (w/v), polysorbate 80 2% (w/v), BAC 0.02% (w/v) in 0.01M citrate buffer pH 4.5.

As noted above, depending on the amount of formulation delivered per pump of a delivery device used to administer the formulation and the desired dose per administration, the octreotide concentration in the formulation is correspondingly adjusted. The concentration may be, for example, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 mg/mL. In some embodiments, the concentration may be from about 1 to about 10 mg/ml. In some embodiments, the concentration may be 1, 2, 3, 4, 5, 6, 7, 8, 9, or 10 mg/mL. In some embodiments, the amount of octreotide delivered per administration (e.g., using a delivery device or pump) is about 1, 5, 10, 20, 30, 40, 50, 60, 70, 80, 90, 100, 150, 200, 250, 300, 350, 400, 450 or 500 microgram (mcg). In some embodiments, the volume of liquid formulation delivered per administration (e.g., using a delivery device or pump) is about 10, 20, 30, 40, 50, 60, 70, 80, 90, 100, 110, 120, 130, 140, 150, 160, 170, 180, 190 or 200 microliters (mcl).

In some embodiments, the liquid formulation is administered once a day (q.d.). In some embodiments the liquid formulation is administered twice a day (b.i.d.). In some embodiments the liquid formulation is administered three times a day (t.i.d.). In some embodiments the liquid formulation is administered more than three times a day (e.g., four times a day, q.i.d.). The dosing frequency, duration, and other aspects of the dosing regimen may be guided by the health care professional to achieve a desired clinical effect.

Any of the foregoing liquid formulations or any other liquid formulations comprising a somatostatin mimetic as described herein, may be provided for delivery using any one of a number of liquid delivery devices, such as but not limited to the VP7-232NE device of Aptar Pharmaceuticals.

In some embodiments, the droplet size of a liquid formulation comprising a somatostatin mimetic disclosed herein may be less than about 120 μm. In some embodiments, the droplet size is between about 15 μm and about 120 μm, between about 15 μm and about 100 μm between about 15 μm and about 80 μm, between about 15 μm and about 70 μm, between about 30 μm and about 70 μm or between about 30 μm to about 40 μm.

In some embodiments, the spray content uniformity of a liquid somatostatin mimetic formulation disclosed herein may be within about 1% relative standard deviation (RSD). In some embodiments, the spray content uniformity of a liquid somatostatin mimetic formulation disclosed herein may be within about 100% of target delivered dose (TDD).

In some embodiments, the pH of a liquid somatostatin mimetic formulation disclosed herein have a pH of about 4.5, a pH of between about 4.0 and about 5.0, a pH of between about 4.0 and about 4.5, a pH of between about 4.5 and 5.0, or a pH of between about 4 and about 6. In some embodiments, the desired buffering pH is achieved using a buffer such as but not limited to citrate, TRIS, HEPES, acetate, phosphate, phosphate-buffered saline, MOPS or MES. The final pH of a formulation is adjusted using an acid or base as needed, such as HCl or NaOH.

In some embodiments, the osmolality of a liquid somatostatin mimetic formulation disclosed herein is between about 290 and 500 mOsm/kg. In some embodiments, the osmolality of the formulation as described herein in increased to within about 290 to about 500 mOsm/kg by the additional of a tonicity agent. Non-limiting examples of tonicity agents include mannitol, dextrose, sodium chloride, sorbitol, maltitol, fructose, or any combination thereof.

In some embodiments, the viscosity of a liquid somatostatin mimetic formulation disclosed herein, η (Pa s) is about 0.4 to about 1.05.

### Powder Formulations

A powder formulation of a somatostatin mimetic such as octreotide may be prepared using excipients such as mannitol, calcium carbonate, lactose, sorbitol, maltitol, L-leucine, lecithin (phosphatidylcholine; e.g. LIPOID S 80, soybean phospholipids with 75% phosphatidylcholine), hydrogenated phosphatidylcholine (e.g., PHOSPHOLIPON 80 H [hydrogenated soybean phospholipids, >70% phosphatidylcholine]), in addition to those described elsewhere herein. Such excipients may be included singly or in any combination or any combination thereof.

In some embodiments, a powder formulation comprises about 0.1 to about 1% octreotide; about 85 to about 99% of a carrier such as mannitol, dextrose, lactose, sorbitol, fructose, or calcium carbonate; and about 1% to about 10% of a phospholipid such as lecithin.

In some embodiments, a powder formulation comprises about 0.1 to about 1% octreotide, about 85 to about 99% calcium carbonate; about 1 to about 5% mannitol, dextrose, lactose, sorbitol, fructose, or any combination thereof; and about 1% to about 10% L-leucine.

In some embodiments, mannitol is provided at about 85 to about 99%. In some embodiments, mannitol is provided at 95%.

In some embodiments, the mannitol is provided at about 1 to about 10%. In some embodiments the mannitol is provided at about 2.5%.

In some embodiments the L-leucine is provide at about 1 to about 10%. In some embodiments, the L-leucine is provided at 2.5%.

In some embodiments the lecithin (e.g., phosphatidyl choline or hydrogenated

phosphatidylcholine) is provided at about 1 to about 10%. In some embodiments, the hydrogenated lecithin is provided at about 2.5%.

In some embodiments, the calcium carbonate is provided at about 80% to about 99%. In some embodiments the calcium carbonate is 95%.

In some embodiments, a mannitol/lecithin formulation comprises 95% mannitol and 5% hydrogenated phosphatidylcholine.

In some embodiments, a calcium carbonate/mannitol/L-leucine formulation comprises 95% calcium carbonate, 2.5% mannitol and 2.5% L-leucine.

In powder formulations, the somatostatin mimetic such as octreotide is provided at about 0.1% to about 1.0%. In some embodiments, the octreotide is 0.1% or 0.5%. As noted above, depending on the amount of formulation delivered per pump of a delivery device used to administer the formulation and the desired dose per administration, the octreotide concentration in the formulation is correspondingly adjusted. The concentration may be, for example, about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1%. In some embodiments, the concentration may be from about 1 to about 10%, In some embodiments, the concentration may be 1, 2, 3, 4, 5, 6, 7, 8, 9, or 10%. In some embodiments, the amount of octreotide delivered per administration (e.g., using a delivery device) is about 1, 5, 10, 20, 30, 40, 50, 60, 70, 80, 90, 100, 150, 200, 250, 300, 350, 400, 450 or 500 microgram (mcg). In some embodiments, the amount of powder formulation delivered per administration (e.g., using a delivery device) is about 1, 5, 10, 20, 30, 40, 50, 60, 70, 80, 90, 100, 110, 120, 130, 140, 150, 160, 170, 180, 190 or 200 milligrams (mg). In some embodiments, the amount of powder formulation delivered per administration (e.g., using a delivery device) is about 1, 5, 10, 20, 30, 40, 50, 60, 70, 80, 90, 100, 110, 120, 130, 140, 150, 160, 170, 180, 190 or 200 mm3 (cubic mm).

In some embodiments, the powder formulation is administered once a day (q.d.). In some embodiments the powder formulation is administered twice a day (b.i.d.). In some embodiments the powder formulation is administered three times a day (t.i.d.). In some embodiments the powder formulation is administered more than three times a day (e.g., four times a day, q.i.d.). The dosing frequency, duration, and other aspects of the dosing regimen may be guided by the health care professional to achieve a desired clinical effect.

Non-limiting examples of powder formulations useful for the purposes disclosed herein include the following (in w/w of powder formulation). Each formulation was spray dried at either 100° C. or 120° C. to evaluate the resulting powder (see Examples).
Octreotide 0.1%, mannitol 95%, lecithin 5%.
Octreotide 0.5%, mannitol 95%, lecithin 5%.
Octreotide 0.1%, calcium carbonate 95%, mannitol 2.5%, L-leucine 2.5%
Octreotide 0.5%, calcium carbonate 95%, mannitol 2.5%, L-leucine 2.5%

Such powder formulation are prepared from a liquid formulation then made into a powder by methods such as lyophilization and spray drying. The material may be further milled, agglomerated, pulverized, sieved, etc. to achieve a powder characteristic compatible with a powder delivery device.

As will be seen in the examples below, liquid and powder formulations of octreotide were developed that will deliver the requisite dose by nose-to-brain delivery for the uses herein described. The European Pharmacopoeia HPLC method for Octreotide quantification was used to study liquid and powder formulations of octreotide for nose-to-brain delivery.

For the liquid formulation, results for the Droplet Size Distribution Dv (50) values at 3 cm, for Formulation 1 ranged from 33.25-35.88 μm, Formulation; Formulation 2 ranged from 32.98-37.5 μm, Formulation 3 ranged from 31.71-33.32 μm and Formulation 4 ranged from 31.92-32.98 μm. The Dv 50 values at 6 cm, for Formulation 1 ranged from 38.96-41.60 μm, Formulation 2 ranged from 38.16-41.93 μm, Formulation 3 ranged from 35.66-39.36 μm and Formulation 4 ranged from 37.29-41.97 μm. The Spray Content Uniformity was performed with each formulation with an n=10. Recovery for all Formulations were very close to the Target Delivered Doses of either 10 or 100 μg. pH measurements were very similar for all formulations and within the pH range for the nasal cavity. Osmolality results showed the Avicel containing formulations 1 and 2 had values of 31.33 and 32.67 mOsm, while the PEG 400 containing formulations 3 and 4 had values of 83.67 and 80.00 mOsm; which tonicities can be increased using any one of more of a number of agents. Viscosity testing were also completed with the Avicel containing formulations showed increased viscosity, where the PEG 400 containing formulations showed very little change in viscosity.

For the powder formulations, all 8 formulations have been successfully spray dried and generated suitable yields of recovery. All powders have been characterized using in-vitro tools. For TGA: all powders showed low/negligible levels of moisture. For SCU, Delivered Doses were consistent for all formulations.

As noted above, depending on the amount of formulation delivered per insufflation of a delivery device used to administer the formulation, the octreotide concentration in the formulation is correspondingly adjusted.

Delivery Devices. Notwithstanding the disclosure herein of a liquid or powder formulation comprising a somatostatin mimetic such as octreotide, a formulation disclosed herein is typically used in combination with a delivery device to deliver the liquid, gel, or powder formulation into the nose and ideally to the olfactory region. In some embodiments, the methods and formulations disclosed herein are achieved and delivered using such a delivery device.

Not wishing to be bound by theory and for which no duty of disclosure is necessary, nasal spray drug products contain therapeutically active pharmaceutical ingredients (API; e.g. octreotide) dissolved or suspended in non-pressurized dispensers that deliver a spray containing a metered dose of the API. The dose can be metered by the spray pump or pre-metered during manufacture. Upon actuation of the dose from nasal spray systems, the spray events can be divided into three phases: (1) formation, (2) fully developed/stable, and (3) dissipation. In the formation phase, flow through the spray pump nozzle is relatively low, droplet size is large, and the output of the nasal spray product is not yet stable. Flow is also low during dissipation at the end of the spray event when the metering chamber is empty. Nasal solution or suspension drug products consist of API particles either dissolved or suspended in an aqueous system in the presence of a range of different excipients, respectively. These excipients can range from preservatives, viscosity modifiers, emulsifiers and buffering agents. For suspension nasal products, the API particle size is a key critical quality attribute, which will affect emitted API particle size and regional deposition of API in the nose. In addition, the particle size of the API will affect the rate of dissolution and permeability at site of deposition in the nasal epithelium and thereby systemic exposure of the API from the nose. Other physicochemical properties of the API such as polymorphic form, solvated/hydrated form, rugosity and surface properties may also affect the rate of API dissolution and therefore affect systemic exposure from the nose. In the case of peptide formulations careful characterization of the molecule is undertaken to ensure drug product quality requirements are met. For both solution and suspension nasal sprays droplet size and rheological properties of the formulation will impact the regional deposition. Such characterization is described herein in the Examples below, such that the formulations described herein achieve the desired features for efficacious nose-to-brain delivery for successfully treating the conditions disclosed herein.

In some embodiments, droplet sizes of 50 μm favored anterior deposition (mainly at the nasal vestibule), and particles of this size are unable to flow freely within the nasal perplexed anatomy. In some embodiments, droplets within the 2.6-14.3 μm particle size range and 10-11 μm diameter achieve maximum deposition in the central nasal areas. Rheological properties influence spray droplet sizes are known to impact on nasal regional deposition. Agents such as but not limited to pectin, gellan gum, and sodium hyaluronate have been used as in situ gelling system to develop a nasal suspension formulations. These agents have shown to increase turbinate deposition posterior to the nasal valve. Such gelling agents may create thixotropic formulation systems that remain gel like at rest but produce fine droplets on shear thinning when the pump is actuated. In some embodiments, thixotropic formulations containing hypromellose are able to achieve 84% turbinate deposition. While, in some embodiments, viscosity is confused with rheology of nasal formulations, when just the viscosity component of the formulation is increased there is generally decreased deposition into the turbinate and olfactory regions of the nose.

In one embodiment, Aptar Pharma manufactures the UDS (solution or powder formulation) single shot nasal unidose system. In the Examples herein, this system has been selected for the feasibility study based on its key features which make it designed to enable the systemic delivery of drugs without the need for injection or administration by a healthcare professional but has the ability to be administered by a second party. Primeless, with one handed actuation and 360° Functionality. This system has a liquid dose up to 100 μL in volume or 140 mm3 powder dosage. In some embodiments, the UDS system is used with a liquid or powder formulation disclosed herein to achieve the purposes hereof.

As shown in the Examples herein, in developing a solution formulation of octreotide, the solubility of the compound under pH range of 4-8 is desired, in one embodiment, since this pH is well tolerated in the nasal cavity. In some embodiments, the addition of dextran, sorbitol, fructose, dextrose, mannitol or sodium chloride modulates the tonicity and viscosity of the formulation together with other buffering agents. In some embodiments, an osmolality target for the formulation is about 290 mOsm/kg to prevent damage to the nasal cilia. In some embodiments the osmolality of the formulation is between about 290 and about 500 mOsm/kg.

In one embodiment, a powder formulation system is prepared by co-spray drying a solution of octreotide in the presence of calcium carbonate, leucine and mannitol. In one embodiment, a powder formulation system is prepared by co-spray drying a solution of octreotide in the presence of mannitol and lecithin. In one embodiment, a powder formulation system is prepared by co-spray drying a solution of octreotide in the presence of trimethyl-chitosan, leucine and mannitol. In one embodiment, such construct creates a powder formulation with high dispersibility and contain absorption enhancer within the matrix. The density of powder should be between 0.6-0.8 g/mL which will enable a wide range of doses that could be filled into the UDS/P system.

### Other Formulations

Descriptions of some such systems are provided herein but are not intended in any way to be limiting as to the selection of the formulation and/or device used for intranasal delivery. Reference to patent and publications is intended merely as a guide. Each and every reference herein is incorporated herein by reference.

### Other Intranasal Formulations

A somatostatin mimetic such as but not limited to those described above may be provided for administration in any formulation compatible with intranasal administration including direct nose-to-brain administration. The intranasal formulation may be a powder, a liquid, a gel, or any other form that achieves the intranasal delivery of a somatostatin mimetic as described herein. IIH and cluster headache are among conditions and diseases treatable by agonists or ligands of one or more somatostatin receptor subtypes 1-5, and wherein intranasal administration offers a facile and patient-compliant means for dosing.

Alkylglycosides and related compounds can be used to enhance intranasal delivery of peptides. U.S. Pat. No. 8,833,728 (to Aegis Therapeutics LLC) describes a pharmaceutical compositions and methods for delivering a polypeptide to the central nervous system of a mammal via intranasal administration. Nasal absorption is enhanced using, for example, an aqueous composition comprising a therapeutic polypeptide and a compound such as 1-O-n-dodecyl-beta-D-maltopyranoside, 1-O-n-decyl-beta-D-maltopyranoside, 1-O-n-tetradecyl-beta-D-maltopyranoside, and beta-D-fructopyranosyl-alpha-glucopyranoside monododecanoate. In some embodiments, the compound is present in the aqueous composition at a concentration between 0.125 g to 1 g per 100 ml of the aqueous composition. U.S. Pat. No. 10,046,025 (also to Aegis Therapeutics LLC) describes an intranasal formulation comprising dodecyl-β-D-maltoside. Other patents include U.S. Pat. Nos. 7,998,927, 8,133,863, 8,226,949, EP EP18731691.4 and U.S. Ser. No. 16/615,362, all of which are incorporated herein by reference. Additional intranasal formulations and delivery systems are described in Wang et al, 2019, ibid, which is incorporated herein by reference in its entirety.

Formulations comprising a somatostatin mimetic in a liposomal particle including nanoparticles are embraced herein. Such formulations are described by Ong et al., Nose-to-brain drug delivery by nanoparticles in the treatment of neurological disorders, Curr Med Chem. 2014;21(37):4247-56; Ghadiri et al., Strategies to Enhance Drug Absorption via Nasal and Pulmonary Routes, Pharmaceutics 2019, 11, 113; by way of non-limiting example.

The present disclosure also embraces somatostatin mimetic formulations used for routes of delivery other than intranasal, such as but not limited to intravenous, intramuscular, subcutaneous, and oral. In some embodiments, a sustained release or depot formulation of a somatostatin mimetic is utilized for intranasal administration as described here. Non-limiting examples of such formulations include microspheres described in U.S. Pat. Nos. 7,399,486, 9,877,922, and 9,155,702 (to Peptron Inc.); other formulations are described elsewhere herein.

Other examples of intranasal formulations are described for particular nasal delivery systems and devices, as described further below. Such intranasal formulations may be used together with the particular delivery system or device as described, or may be used with a different delivery device including devices such as a nasal spray, pumps, insufflators or atomizers.

### Intranasal Delivery Devices

Nasal delivery devices including nose-to-brain delivery devices, and their respective formulations of a somatostatin mimetic, are useful to achieve the purposes disclosed herein. A somatostatin mimetic such as but not limited to those described above may be provided for administration using any device compatible with intranasal administration, and on one aspect, for nose-to-brain delivery.

U.S. Pat. No. 10,507,295 (to Impel NeuroPharma Inc) describes a device for delivery of a compound to an olfactory region of a nasal cavity, the device comprising: an actuator body comprising a vertical portion and an angled portion, the vertical portion comprising a contoured surface to accommodate a facial component of a user's face and configured to house a pressurized propellant container containing propellant and a first portion of a connection channel, the first portion of the connection channel configured to be in communication with the propellant container, the angled portion comprising an additional contoured surface to accommodate an additional component of the user's face and configured to house a second portion of the connection channel; a diffuser in communication with the second portion of the connection channel; a drug chamber in communication with the diffuser, the drug chamber configured to hold the compound; a nozzle in communication with the drug chamber, wherein propellant released from the propellant container is configured to travel through the connection channel, contact the diffuser, and propel the compound out the nozzle forming a plume.

U.S. Pat. No. 8,978,647 (to OptiNose AS) describes a nasal delivery device for delivering substance to a nasal cavity of a subject, the delivery device comprising: a nosepiece for fitting to a nostril of a subject, wherein the nosepiece comprises a tip element which includes a delivery aperture from which substance is in use delivered into the nasal cavity, and the tip element is at least in part tapered so as to be inclined to a longitudinal axis of the nosepiece, with the delivery aperture extending both laterally across the tip element and along a longitudinal extent of the tip element in relation to the longitudinal axis, wherein the tip element of the nosepiece has differing lengths adjacent forwardmost and rearwardmost sections thereof; a nozzle through which substance is in use delivered to the respective nasal cavity; and a delivery unit for delivering substance through the nozzle of the nosepiece.

U.S. Pat. No. 7,841,337 (to OptiNose AS) describes an Exhalation Delivery System (EDS) actuated by breath, wherein a mouthpiece through which a user in use exhales to actuate the delivery device; a nosepiece for fitting to a nostril of the user through which a substance is in use delivered; a substance supply unit actuatable to deliver a dose of a substance through the nosepiece; a loading unit operable to load the substance supply unit with an actuation force; and a release mechanism for enabling actuation of the substance supply unit in response to exhalation by the user through the mouthpiece; wherein the release mechanism comprises a locking unit which is movable between a locking configuration in which the substance supply unit is locked in a non-actuated position when loaded by the loading unit and a release configuration in which the substance supply unit is actuatable by the loading unit, and a trigger member for releasing the locking unit from the locking configuration to the release configuration in response to exhalation by the user through the mouthpiece and thereby enabling actuation of the substance supply unit.

U.S. Pat. No. 10,238,577 (to Zeteo Biomedical LLC) describes devices for delivery of fluid compositions include a delivery device including an internally pierced blister in which the device includes a plunger configured to crush the blister and deliver the contents in a lateral direction with respect to the motion of a dispensing button. Such devices can be used for intranasal delivery. International patent publication WO2020198536 (to Zeteo Biomedical, LLC) describes a handheld assembly for dispensing a medicament to a subject. The assembly includes a unit dose device, a shell, a plunger, a dispense button, a drive member and an escapement that is movable and capable of cycling the dispense assembly thru multiple states of a dispense cycle. Intranasal delivery including nose-to-brain delivery are described.

Another example of an intranasal delivery system comprises pressurized compartment containing medicament and excipient, pre-loaded into a lightweight casing, wherein finger-tip snap compression of the casing results in cyclonic motion for accurate drug release of powdered formulations; see U.S. Pat. Nos. 7,163,013 and 8,695,592 (to Alchemy Pharmatech Ltd).

Any of the devices and formulations described herein may be used for the purposes disclosed herein. Other formulations and devices to achieve effective intranasal delivery are described below. All of the citations herein are incorporated herein by reference in their entireties.

In some embodiments, the intranasal formulation and delivery system comprises an exhalation delivery system such as described in U.S. Pat. Nos. 10,076,614, 10,076,615, 10,124,132, and 10,179,216, incorporated herein in their entireties.

In some embodiments, the intranasal formulation and delivery system comprises a delivery system as described in any one or more of U.S. Pat. Nos. 10,765,829; 10,737,045; 10,722,667; 10,695,295; 10,682,414; 10,653,745; 10,639,438; 10,639,437; 10,549,052; 10,525,218; 10,478,574; 10,478,405; 10,456,377; 10,420,750; 10,406,200; 10,398,859; 10,300,229; 10,286,164; 10,252,010; 10,220,097; 10,179,216; 10,144,776; 10,124,132; 10,112,021; 10,099,024; 10,099,019; 10,076,615; 10,076,614; 10,066,017; 9,962,397; 9,949,989; 9,949,923; 9,855,247; 9,833,504; 9,744,210; 9,682,205; 9,649,456; 9,629,894; 9,566,402; 9,556,260; 9,522,243; 9,468,727; 9,452,272; 9,339,617; 9,320,800; 9,308,234; 9,308,191; 9,272,104; 9,249,424; and US patent publication 20190269867, all of which are incorporated by reference herein.

In some embodiments, the intranasal formulation and delivery system comprises a delivery system as described in any one or more of U.S. Pat. Nos. 10,653,690; 10,617,682; 10,549,052; 10,292,948; 10,213,487; 10,085,937; 9,775,838; 9,707,226; 9,629,965; 9,561,177; 9,480,644; 9,468,747; 9,211,253; 9,033,939; 8,567,390; , all of which are incorporated by reference herein.

The foregoing are merely non-limiting examples of nasal formulations and nasal delivery systems that can be used to deliver the somatostatin mimetic for the purposes disclosed herein.

### Unit Dose

Unit dose refers to the amount of somatostatin mimetic (dose amount) delivered into the nose per single prescribed time of administration (e.g., one insufflation in each nostril, two insufflations in each nostril, etc.). As noted herein, the unit dose of the somatostatin mimetic in the intranasal formulation is provided such that the intranasal formulation is effective in the treatment of any condition or disease described herein, and moreover, is effective at a dose level and dosing regimen (e.g., dose level, frequency of daily administration, duration of dosing in days or longer time periods, the time of start and ending of a course of a dosing regimen and cycles of a dosing regimen, among others) that is lower than the dose and dosing regimen of the same somatostatin mimetic administered by a route other than intranasal for the treatment of the conditions and diseases described herein, or lower than the dose and dosing regimen of the same somatostatin mimetic administered intranasally for the treatment of conditions and diseases other than described herein. The unit dose may be prescribed for administration one time or multiple times per day, e.g., one insufflation in each nostril, three times a day; two insufflations in each nostril twice a day, etc.). Dosing regimen refers to the dose administration factors such as frequency of daily dosing (e.g., once [q.d.], twice [b.i.d], thrice [t.i.d], four times [q.i.d], five times and so forth, including every other day, every third day, twice a week, weekly, and so forth); duration of administration (e.g., daily for one or more days, one week or more than one week, one month or more than one month, chronically, and any combination or components of any of the foregoing); cycles of dosing with periods of no dosing, such as one week of dosing with a one week no-dosing period, and repeated; two week cycles with a week or two weeks of non-dosing in between, etc.). In some instances, dosing regimen may include the dose (dose level). Such dosing regimens may be provided for treating an acute episode as well as for providing maintenance therapy to prevent or reduce recurrence. In some embodiments, the maintenance dose will be of a lower dose level, a reduced dosing frequency, or the combination of both, to reduce or protect from recurrence.

The intranasal formulation and/or delivery device may comprise a single unit dose packaging of the somatostatin mimetic, or may be provided in multiple unit dose packaging. Such format of packaging will be provided for facile storage of the formulation and/or device, and provide for facile administration depending on the prescribed dosing regimen.

In some embodiments, the dose and dosing regimen described herein provides for an effective administration that is lower (e.g., lower total daily dose) than the subcutaneous dose and dosing regimen of the same somatostatin mimetic. In some embodiments, the dose and dosing regimen described herein provides for an effective administration that is lower (e.g., lower total daily dose) than the intravenous dose and dosing regimen of the same somatostatin mimetic. In some embodiments, the dose and dosing regimen described herein provides for an effective administration that is lower (e.g., lower total daily dose) than the intramuscular dose and dosing regimen of the same somatostatin mimetic. In some embodiments, the dose and dosing regimen described herein provides for an effective administration that is lower (e.g., lower total daily dose) than the deep subcutaneous dose and dosing regimen of the same somatostatin mimetic. In some embodiments, the dose and dosing regimen described herein provides for an effective administration that is lower (e.g., lower total daily dose) than the depot dose and dosing regimen of the same somatostatin mimetic. In some embodiments, the dose and dosing regimen described herein provides for an effective administration that is lower (e.g., lower total daily dose) than the oral dose and dosing regimen of the same somatostatin mimetic. In any of the foregoing embodiments, the lower dose and dosing regimen is for treating the same condition or disease.

Moreover, the efficacious dose of a somatostatin mimetic for the conditions and diseases described herein and in general those benefitted by the direct nose-to-brain delivery of a somatostatin mimetic, is substantially lower than the dose required by a nasal route of administration that is not nose-to-brain administration or not formulated for nose-to-brain administration.

In some embodiments, the dose and dosing regimen described herein provides for an effective administration that is lower (e.g., lower total daily dose) than the intranasal dose and dosing regimen of the same somatostatin mimetic for treating other conditions and disease than those described herein, such as acromegaly and carcinoid syndrome. Such intranasal somatostatin mimetic pharmaceutical products have not been approved for treatments of these other diseases.

In some embodiments, the systemic exposure achieved by the intranasal administration of a somatostatin mimetic, and in particular the direct nose-to-brain route, as disclosed herein is lower than the systemic exposure achieved by another route of administration such as a parenteral (e.g., intravenous, subcutaneous, intradermal, intramuscular, intraperitoneal) or oral route. In some embodiments, the side effect profile and/or adverse events elicited by the intranasal administration of a somatostatin mimetic, and in particular the direct nose-to-brain route, as disclosed herein is lower than the side effect profile and/or adverse events achieved by another route of administration such as a parenteral (e.g., intravenous, subcutaneous, intradermal, intramuscular, intraperitoneal) or oral route. In some embodiments, systemic absorption of the somatostatin mimetic is essentially avoided by the intranasal administration, and in particular the direct nose-to-brain route, as disclosed herein.

In some embodiments, the systemic exposure achieved by direct nose-to-brain administration of a somatostatin mimetic, as disclosed herein is lower than the systemic exposure achieved by the traditional intranasal route of administration.

In some embodiments, the dose of effective intranasal amount of somatostatin mimetic is equal to or less than about 300 mcg per day, equal to or less than about 200 mcg per day, equal to or less than about 150 mcg per day, equal to or less than about 100 mcg per day, equal to or less than about 90 mcg per day, equal to or less than about 80 mcg per day, equal to or less than about 70 mcg per day, equal to or less than about 60 mcg per day, equal to or less than about 50 mcg per day, equal to or less than about 40 mcg per day, equal to or less than about 30 mcg per day, equal to or less than about 20 mcg per day, equal to or less than about 10 mcg per day, equal to or less than about 5 mcg per day, or greater than or equal to about 1 mcg per unit dose. In some embodiments, the foregoing doses are administered once a day. In some embodiments, a foregoing dose is administered as a divided dose twice a day. In some embodiments, a foregoing dose is administered as a divided dose three times a day. In some embodiments, a foregoing dose is administered as a divided dose four times a day. In some embodiments, a foregoing dose is administered every other day. For any of the aforementioned doses, less than may extend to the next lower disclosed dose, or to any of the lower disclosed doses described. By way of example, less than or equal to about 60 mcg per day also refers to about 5-60, about 10-60, about 20-60, about 30-60, about 40-60 or about 50-60 mcg per day.

In some embodiments, the serum area-under-the-curve after a single dose of an effective dosing regimen of a somatostatin mimetic administered by the intranasal route is lower than the serum area-under-the-curve after a single dose of an effective dosing regimen of the somatostatin mimetic administered by a route other than intranasal, such as but not limited to subcutaneous, intramuscular, oral and intravenous.

In some embodiments, the serum area-under-the-curve after a single dose and dosing regimen described herein provides for an effective administration that is lower than the serum area-under-the-curve from a subcutaneous dose and dosing regimen of the same somatostatin mimetic. In some embodiments, the serum area-under-the-curve from the dose and dosing regimen described herein provides for an effective administration that is lower than the serum area-under-the-curve from the intravenous dose and dosing regimen of the same somatostatin mimetic. In some embodiments, the serum area-under-the-curve from the dose and dosing regimen described herein provides for an effective administration that is lower than the serum area-under-the-curve from the intramuscular dose and dosing regimen of the same somatostatin mimetic. In some embodiments, the serum area-under-the-curve from the dose and dosing regimen described herein provides for an effective administration that is lower than the serum area-under-the-curve from the deep subcutaneous dose and dosing regimen of the same somatostatin mimetic. In some embodiments, the serum area-under-the-curve from the dose and dosing regimen described herein provides for an effective administration that is lower than the serum area-under-the-curve from the depot dose and dosing regimen of the same somatostatin mimetic. In some embodiments, the serum area-under-the-curve from the dose and dosing regimen described herein provides for an effective administration that is lower than the serum area-under-the-curve from the oral dose and dosing regimen of the same somatostatin mimetic. In any of the foregoing embodiments, the lower dose and dosing regimen is for treating the same condition or disease.

In some embodiments, the serum area-under-the-curve of the intranasal dose of the somatostatin mimetic is about equal to or less than about 50% of the serum area-under-the-curve of the somatostatin mimetic administered by a non-intranasal route. In some embodiments, the serum area-under-the-curve of the intranasal dose of the somatostatin mimetic is about equal to or less than about 45% of the serum area-under-the-curve of the somatostatin mimetic administered by a non-intranasal route. In some embodiments, the serum area-under-the-curve of the intranasal dose of the somatostatin mimetic is about equal to or less than about 40% of the serum area-under-the-curve of the somatostatin mimetic administered by a non-intranasal route. In some embodiments, the serum area-under-the-curve of the intranasal dose of the somatostatin mimetic is about equal to or less than about 35% of the serum area-under-the-curve of the somatostatin mimetic administered by a non-intranasal route. In some embodiments, the serum area-under-the-curve of the intranasal dose of the somatostatin mimetic is about equal to or less than about 30% of the serum area-under-the-curve of the somatostatin mimetic administered by a non-intranasal route. In some embodiments, the serum area-under-the-curve of the intranasal amount of the somatostatin mimetic is about equal to or less than about 25% of the serum area-under-the-curve of the somatostatin mimetic administered by a non-intranasal route. In some embodiments, the serum area-under-the-curve of the intranasal dose of the somatostatin mimetic is about equal to or less than about 20% of the serum area-under-the-curve of the somatostatin mimetic administered by a non-intranasal route. In some embodiments, the serum area-under-the-curve of the intranasal dose of the somatostatin mimetic is about equal to or less than about 15% of the serum area-under-the-curve of the somatostatin mimetic administered by a non-intranasal route. In some embodiments, the serum area-under-the-curve of the intranasal amount of the somatostatin mimetic is about equal to or less than about 10% of the serum area-under-the-curve of the somatostatin mimetic administered by a non-intranasal route. In some embodiments, the serum area-under-the-curve of the intranasal dose of the somatostatin mimetic is about equal to or less than about 5% of the serum area-under-the-curve of the somatostatin mimetic administered by a non-intranasal route. In some embodiments, the serum area-under-the-curve of the intranasal dose of the somatostatin mimetic is about equal to or less than about 4%, 3%, 2% or 1% of the serum area-under-the-curve of the somatostatin mimetic administered by a non-intranasal route. For any of the aforementioned percentages, less than may extend to the next lower disclosed percentage, or to any of the lower disclosed percentages described. By way of example, less than or equal to about 25% also refers to about 1-25%, about 2-25%, about 3-25%, about 4-25%, about 5-25%, about 10-25%, about 15-25%, or about 20-25%.

In some embodiments, the serum area-under-the-curve from a single intranasal dose of a somatostatin mimetic is equal to or less than about 900 mcg L−1 min. In some embodiments, the serum area-under-the-curve from a single intranasal dose of a somatostatin mimetic is equal to or less than about 800 mcg L−1 min. In some embodiments, the serum area-under-the-curve from a single intranasal dose of a somatostatin mimetic is equal to or less than about 700 mcg L−1 min. In some embodiments, the serum area-under-the-curve from a single intranasal dose of a somatostatin mimetic is equal to or less than about 600 mcg L−1 min. In some embodiments, the serum area-under-the-curve from a single intranasal dose of a somatostatin mimetic is equal to or less than about 500 mcg L−1 min. In some embodiments, the serum area-under-the-curve from a single intranasal dose of a somatostatin mimetic is equal to or less than about 400 mcg L−1 min. In some embodiments, the serum area-under-the-curve from a single intranasal dose of a somatostatin mimetic is equal to or less than about 300 mcg L−1 min. In some embodiments, the serum area-under-the-curve from a single intranasal dose of a somatostatin mimetic is equal to or less than about 200 mcg L−1 min. In some embodiments, the serum area-under-the-curve from a single intranasal dose of a somatostatin mimetic is equal to or less than about 100 mcg L−1 min. In some embodiments, the serum area-under-the-curve from a single intranasal dose of a somatostatin mimetic is equal to or less than about 90 mcg L−1 min. In some embodiments, the serum area-under-the-curve from a single intranasal dose of a somatostatin mimetic is equal to or less than about 80 mcg L−1 min. In some embodiments, the serum area-under-the-curve from a single intranasal dose of a somatostatin mimetic is equal to or less than about 70 mcg L−1 min. In some embodiments, the serum area-under-the-curve from a single intranasal dose of a somatostatin mimetic is equal to or less than about 60 mcg L−1 min. In some embodiments, the serum area-under-the-curve from a single intranasal dose of a somatostatin mimetic is equal to or less than about 50 mcg L−1 min. In some embodiments, the serum area-under-the-curve from a single intranasal dose of a somatostatin mimetic is equal to or less than about 40 mcg L−1 min. In some embodiments, the serum area-under-the-curve from a single intranasal dose of a somatostatin mimetic is equal to or less than about 30 mcg L−1 min. In some embodiments, the serum area-under-the-curve from a single intranasal dose of a somatostatin mimetic is equal to or less than about 20 mcg L−1 min. In some embodiments, the serum area-under-the-curve from a single intranasal dose of a somatostatin mimetic is equal to or less than about 10 mcg L−1 min. In some embodiments, the serum area-under-the-curve from a single intranasal dose of a somatostatin mimetic is equal to or less than about 5 mcg L−1 min. For any of the aforementioned AUCs, less than may extend to the next lower disclosed AUC, or to any of the lower disclosed AUCs described. By way of example, less than or equal to about 30 mcg L−1 min also refers to about 5-30, about 10-30 or about 20-30 mcg L−1 min.

In some embodiments, the serum area-under-the-curve after a single dose of an effective dosing regimen of a somatostatin mimetic administered by the intranasal route is lower than the serum area-under-the-curve after a single dose of an effective dosing regimen of the somatostatin mimetic administered for another indication by the intranasal route. Non-limiting examples of other indications include acromegaly and carcinoid syndrome.

In some embodiments, the serum area-under-the-curve of the intranasal amount of the somatostatin mimetic is about equal to or less than about 50% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 45% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 40% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 35% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 30% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 25% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 20% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 15% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 10% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 5% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 4%, 3%, 2% or 1% of the serum area-under-the-curve of the somatostatin mimetic administered by the intranasal route for an indication other than IIH or cluster headache. For any of the aforementioned percentages, less than may extend to the next lower disclosed percentage, or to any of the lower disclosed percentages described. By way of example, less than or equal to about 25% also refers to about 1-25%, about 2-25%, about 3-25%, about 4-25%, about 5-25%, about 10-25%, about 15-25%, or about 20-25%.

Thus, a pharmaceutical composition in unit dose form may comprise about equal to or less than about 300 mcg per unit dose, equal to or less than about 250 mcg per unit dose, equal to or less than about 200 mcg per unit dose, equal to or less than about 100 mcg per unit dose, equal to or less than about 90 mcg per unit dose, equal to or less than about 80 mcg per unit dose, equal to or less than about 70 mcg per unit dose, equal to or less than about 60 mcg per unit dose, equal to or less than about 50 mcg per unit dose, equal to or less than about 40 mcg per unit dose, equal to or less than about 30 mcg per unit dose, equal to or less than about 20 mcg per unit dose, equal to or less than about 10 mcg per unit dose or equal to or less than about 5 mcg per unit dose, and greater than or equal to about 1 mcg per unit dose. For any of the aforementioned unit doses, less than may extend to the next lower disclosed unit dose, or to any of the lower disclosed unit doses described. By way of example, less than or equal to about 80 mcg per unit dose also refers to about 1-80, 5-80, about 10-80, about 20-80, about 30-80, about 40-80 or about 50-80 mcg per unit dose.

The following embodiments, while non-limiting, are examples of certain aspects of the disclosure. In some embodiments, a method is provide for treating idiopathic intracranial hypertension (IIH) or cluster headache in a subject in need thereof, comprising administering to the subject an effective intranasal amount of a somatostatin mimetic formulated for intranasal administration. In some embodiment, the intranasal administration is direct nose-to-brain administration.

In some embodiments, use of an intranasal formulation of a somatostatin mimetic is provided for intranasal administration for treating idiopathic intracranial hypertension (IIH) or cluster headache. In some embodiments, intranasal administration is direct nose-to-brain administration.

In some embodiments, the effective intranasal amount is less than the amount effective when the somatostatin mimetic is administered by a non-intranasal route in accordance with the same dosing regimen. In some embodiments, a serum area-under-the-curve from a unit dose of the intranasal amount of the somatostatin mimetic is less than the serum area-under-the-curve of an effective unit dose of the somatostatin mimetic administered by a non-intranasal route. In some embodiments, the non-intranasal route is subcutaneous, intramuscular, oral or intravenous.

In some embodiments, a serum area-under-the-curve from a unit dose of the intranasal amount of the somatostatin mimetic is less than the serum area-under-the-curve from an effective unit dose of the somatostatin mimetic administered by the intranasal route for an indication other than IIH or cluster headache. In some embodiments, the indication other than IIH or cluster headache is acromegaly or carcinoid syndrome.

In some embodiments, the effective intranasal amount of a somatostatin mimetic is about equal to or less than about 100 mcg daily. In some embodiment, the serum area-under-the-curve of the intranasal amount of the somatostatin mimetic is about equal to or less than about 50% of the serum area-under-the-curve of the somatostatin mimetic administered by a non-intranasal route. In some embodiments, the serum area-under-the-curve of the intranasal amount of the somatostatin mimetic is about equal to or less than about 25% of the serum area-under-the-curve of the somatostatin mimetic administered by a non-intranasal route. In some embodiments, the serum area-under-the-curve of the intranasal amount of the somatostatin mimetic is about equal to or less than about 10% of the serum area-under-the-curve of the somatostatin mimetic administered by a non-intranasal route. In some embodiments, the non-intranasal route is subcutaneous, intramuscular, oral or intravenous.

In some embodiments, the serum area-under-the-curve of the intranasal amount of the somatostatin mimetic is about equal to or less than about 50% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 25% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 10% of the serum area-under-the-curve of the somatostatin mimetic administered by the intranasal route for an indication other than IIH or cluster headache. In some embodiments, the indication other than IIH or cluster headache is acromegaly or carcinoid syndrome.

In some embodiments, the effective intranasal amount is administered as a single daily dose or as 2, 3 or 4 divided doses. In some embodiments, the effective intranasal amount is administered daily for a duration effective to treat or resolve the IIH or cluster headache. In some embodiments, treating is to reduce or prevent an acute, ongoing episode, and/or prophylactic and/or maintenance therapy to prevent future episodes. In some embodiments, the effective intranasal amount of somatostatin mimetic provides a minimal effective dose.

In some embodiments, the somatostatin mimetic is selected from somatostatin, octreotide, lanreotide, pasireotide, pentetreotide, or any combination thereof. In some embodiments, the somatostatin mimetic formulated for intranasal administration comprises a powder, liquid or gel.

In some embodiments, a pharmaceutical composition is provided in unit dose form comprising a somatostatin mimetic formulated for intranasal administration suitable for treatment of idiopathic intracranial hypertension (IIH) or cluster headache. In some embodiments, the intranasal administration is direct nose-to-brain administration. In some embodiments, a pharmaceutical composition is provided in unit dose form comprising a somatostatin mimetic formulated for intranasal administration suitable for use in treatment of idiopathic intracranial hypertension (IIH) or cluster headache. In some embodiments, the intranasal administration is direct nose-to-brain administration.

In some embodiments, the unit dose comprising the somatostatin mimetic contains less somatostatin mimetic than would be effective if administered by a non-intranasal route following the same dosing regimen. In some embodiments, a serum area-under-the-curve of a unit dose of the somatostatin mimetic administered intranasally is less than the serum area-under-the-curve of the unit dose of the somatostatin mimetic administered by a non-intranasal route. In some embodiments, the non-intranasal route is subcutaneous, intramuscular oral or intravenous.

In some embodiments, a unit dose of the somatostatin mimetic formulated for intranasal administration is about equal to or less than about 100 mcg. In some embodiments, a unit dose of the somatostatin mimetic formulated for intranasal administration is about equal to or less than about 50 mcg. In some embodiments, a unit dose of the somatostatin mimetic formulated for intranasal administration is about equal to or less than about 10 mcg. In some embodiments, a unit dose of the somatostatin mimetic formulated for intranasal administration is about equal to or less than about 5 mcg. In some embodiments, a unit dose of the somatostatin mimetic formulated for intranasal administration is equal to about 1 mcg.

In some embodiments, the serum area-under-the-curve of a unit dose of the somatostatin mimetic administered intranasally is about equal to or less than about 50% of the serum area-under-the-curve of a unit dose of the somatostatin mimetic administered by a non-intranasal route. In some embodiments, the serum area-under-the-curve of a unit dose of the somatostatin mimetic administered intranasally is about equal to or less than about 25% of the serum area-under-the-curve of a unit dose of the somatostatin mimetic administered by a non-intranasal route. In some embodiments, the serum area-under-the-curve of a unit dose of the somatostatin mimetic administered intranasally is about equal to or less than about 10% of the serum area-under-the-curve of a unit dose of the somatostatin mimetic administered by a non-intranasal route. In some embodiments, the non-intranasal route is subcutaneous, intramuscular or intravenous.

In some embodiments, a serum area-under-the-curve from a unit dose of the intranasal amount of the somatostatin mimetic is less than the serum area-under-the-curve from an effective single dose of the somatostatin mimetic administered by the intranasal route for an indication other than IIH or cluster headache. In some embodiments, the indication other than IIH or cluster headache is acromegaly or carcinoid syndrome.

In some embodiments, the unit dose is labeled for administration in a single daily dose or as 2, 3 or 4 divided doses. In some embodiments, the unit dose is labeled for daily administration for a duration effective to treat or resolve the IIH or cluster headache. In some embodiments, treatment is to reduce or prevent an acute, ongoing episode, and/or prophylactic and/or maintenance therapy to prevent future episodes. In some embodiments, the unit dose is a minimal effective dose.

In some embodiments, the somatostatin mimetic is selected from somatostatin, octreotide, lanreotide, pasireotide, pentetreotide, or any combination thereof. In some embodiments, the somatostatin mimetic formulated for intranasal administration comprises a powder, liquid or gel.

### EXAMPLES

The following examples are put forth so as to provide persons having ordinary skill in the art with a complete disclosure and description of how to make and use the embodiments disclosed herein, and are not intended to limit the scope of what the disclosure.

### Example 1. Treatment of Idiopathic Intracranial Hypertension

A patient suffering from idiopathic intracranial hypertension is started on a course of treatment with an intranasal formulation of octreotide acetate delivered using a nose-to-brain delivery formulation and device. Within days from the initiation of daily dosing, the patient experiences relief from headache. Intracranial pressure is also found to be reduced. No side effects are associated with the administration.

### Example 2. Treatment of Cluster Headache

A patient suffering from cluster headaches without relief from any therapy during or between episodes is started on a course of treatment with an intranasal formulation of octreotide acetate delivered using a nose-to-brain delivery device. Treatment is started at the onset of a headache cycle. Within one week from the initiation of daily dosing, the patient experiences a reduction in frequency of headaches which breaks the attack cycle. The patient is maintained on a less frequent dosing schedule which prevents recrudescence.

### Example 3. Liquid Formulations of Octreotide for Nose-to-brain Delivery

Liquid formulations of octreotide acetate were prepared for evaluation of droplet size distribution (DSD), spray content uniformity (SCU), pH, osmolality and viscosity, in order to identify a suitable formulation that can be used for nose-to-brain delivery. First, a suitable HPLC system was developed to quantify octreotide. The following table shows the materials being used for this and the ensuing examples.

| Material | Part | Batch | Supplier |
|---|---|---|---|
| Octreotide | HY- 17365/C | Lot# 116521 | MedChemExpress |
| Acetate salt | S- 0944 | | |
| UDSp | Ball | 002982503A | Aptar |
| Device | Container Sub | 0027722920 | Pharma |
| (powder) | Stem Sub | 0028929870 | |
| | Actuator UDSP | 0028928070 | |
| VP7-232NE | Pump | NA | Aptar |
| Devices | TP27.0 mm | | Pharma |
| (liquid) | Bottle | NA | |
| | 20 mL | | |
| | HDPE | | |
| PBS | Excipients | SLCJ5627 | Sigma |
| PEG400 | | 1882895 | Fisher |
| Mannitol | | 1849103 | Fisher |
| Avicel** | | D1936C | DuPont |
| Lecithin*** | | A0414102 | Acros |
| Tween 80 | | BCBW9985 | Sigma |
| Benzalkonium | | BCCD5323 | Sigma |
| chloride (BAC) | | | |

*NA: not applicable.
**Avicel CL-611 is a blend of microcrystalline cellulose and 11.3-18.8% sodium carboxymethycellulose
***or LIPOID S 80, Batch No. 29232000, soybean phospholipids containing 75% phosphatidylcholine

A liquid formulation of octreotide was developed for delivery using a spray device, which has a delivered dose of 10 meg or 100 meg in 100 mcl (microliters). Octreotide formulations comprising 0.1 mg/mL octreotide were prepared for the 10 meg dose in 100 mcl delivered volume, and formulations comprising 1.0 mg/mL octreotide for the 100 meg dose in 100 mcl delivered volume.

The following liquid formulation compositions were prepared for testing in the analyses described, after establishing an HPLC-based analytical method described further below.
Octreotide Formulation 1—Octreotide 0.1 mg/mL, Avicel CL611 2% (w/v), polysorbate 80 2% (w/v), BAC 0.02% (w/v) in 0.01M citrate buffer pH 4.5
Octreotide Formulation 2—Octreotide 1.0 mg/mL, Avicel CL611 2% (w/v), polysorbate 80 2% (w/v), BAC 0.02% (w/v) in 0.01M citrate buffer pH 4.5
Octreotide Formulation 3—Octreotide 1.0 mg/mL, PEG 400 2% (w/v), polysorbate 80 2% (w/v), BAC 0.02% (w/v) in 0.01M citrate buffer pH 4.5
Octreotide Formulation 4—Octreotide 0.1 mg/mL, PEG 400 2% (w/v), polysorbate 80 2% (w/v), BAC 0.02% (w/v) in 0.01M citrate buffer pH 4.5.

### Example 4. Characterization of Liquid Formulations of Octreotide for Nose-to-Brain Delivery

HPLC METHOD. The compendial PhEur HPLC method was used (EP Reference: 01/2020:2414). An Agilent 1260 system with either a MWD or DAD detector is used via Reverse Phase HPLC with a YMC-Pack-ODS-AQ to separate Octreotide via gradient elution. Mobile phases of tetramethyl ammonium hydroxide in different concentrations of water (90% H₂O:10% ACN and 40% H₂O:60% ACN) pH2.0 with a gradient elution at a flow rate of 1.8mL/min was used at a column temperature of 40° C. with an injection volume of 20 μL. UV absorbance was measured at a wavelength of 210 nm over the 50 minute run time. The HPLC parameters are summarized below.

| Parameter | Value to set up | | |
|---|---|---|---|
| HPLC System | Agilent 1260 system, | | |
| and detector | MWD or DAD detector | | |
| Phase | Reverse Phase | | |
| Column | YMC-Pack ODS-AQ, | | |
| | 250 mm × 4.6 mm, 5 μm | | |
| Mobile phase A | Tetramethyl ammonium hydroxide | | |
| | in water for chromatography pH | | |
| | 2.0 900:100 (v/v) acetonitrile; | | |
| Mobile phase B | Tetramethyl ammonium hydroxide | | |
| | in water for chromatography pH | | |
| | 2.0 400:600 (v/v) acetonitrile; | | |
| Flow rate (mL/min) | 1.8 | | |
| Detection | 210 | | |
| Wavelength (nm) | | | |
| Column | 40 | | |
| temperature (° C.) | | | |
| Autosampler | 2-8° C. | | |
| temperature (° C.) | | | |
| Injection volume (μL) | 20 | | |
| Total run time (min) | 50 | | |
| | Time | % | % |
| | (mins) | A | B |
| Gradient elution | 0 | 90 | 10 |
| | 15 | 80 | 20 |
| | 40 | 55 | 45 |
| | 45 | 30 | 70 |
| | 50 | 90 | 10 |

| | |
|---|---|
| Washing Procedure | H2O milliQ 95:5 MeOH |
| | 20 mins 100% ACN |
| | 20 mins |
| | H2O milliQ 50:50 ACN 20 mins |

ACN = acetonitrile

Solution Preparation. All solutions and/or raw active storage, please ensure it is in an airtight container, protected from light, at a temperature of 2° C. to 8° C.

Diluent Preparation. Mix 10 volumes of acetonitrile and 90 volumes of water, then adjust to pH 3.5 with acetic acid. Expiry: 7 days

Mobile Phase A Preparation. Dissolve 4.5 g of tetramethyl ammonium hydroxide in 800 mL of water for chromatography and adjust to pH 2.0 with phosphoric acid; dilute to 900 mL with water for chromatography and add 100 mL of acetonitrile. Expiry: 5 days

Mobile Phase B Preparation. Dissolve 4.5 g of tetramethyl ammonium hydroxide in 300 mL of water for chromatography and adjust to pH 2.0 with phosphoric acid; dilute to 400 mL with water for chromatography and add 600 mL of acetonitrile. Expiry: 5 days

Reference Stock Solution Preparation. Dissolve the contents of a vial of Octreotide CRS in the solvent mixture to obtain a concentration of 1.0 mg/mL.

Linearity, Standard Agreement and System Precision. System suitability was tested to ensure the method was fit for purpose and identified the quantification range. Serial dilutions from stocks solutions were prepared ranging from 1000.00 μg/mL to 0.05 μg/mL in diluent 90% H2O:10% acetonitrile at pH 3.5. Samples prepared above were analyzed by HPLC.

The percentage content of octreotide (C49H66N10O10S2) was calculated taking into account the assigned content of C49H66N10010S2 in Octreotide CRS (European Pharmacopeia octreotide reference standard).

System suitability assessment was as follows:

| Parameter | Acceptance Criteria |
|---|---|
| Limit of Quantification (LOQ) | The Signal to Noise (S/N) ratio for |
| | the LOQ should be NLT 10. |
| Limit of Detection (LOD) | Signal to Noise (S/N) ratio for the |
| | LOD should be NLT 3. |
| Linearity | The correlation co-efficient should |
| | be NLT 0.99. |
| Standard Verification | Between 90-110% |
| Working precision | % RSD NMT 2.0% |
| (Initial 6 WSA injections) | |
| Specificity | PASS if Blank, diluent, Matrix Ingredients |
| | do not interfere with the main peak. |
| Tailing factor | NMT 2.0 |

Results. The system specificity was tested against the blank diluent and the excipients and no interference was seen with the main peak. Octreotide could be identified at a retention time of 20.8 minutes. The standard verification (98.5%) was also well within the required range, as was the working standard precision (0.1% RSD). The linearity showed a R² value of 0.9999, and the limits of quantification and detection had signal to noise ratios of 17.4 and 6.0, respectively. Peak tailing factor was around 1.4 which was below the criteria set of 2.0.

To ensure that the addition of formulation excipients do not interfere with the quantification of the API, samples of excipients were prepared in the standard diluent at the concentration of 1 mg/mL. These were run alongside the usual system suitability sequence using the same HPLC method used for the linearity testing. There was no interference from the diluent. None of the excipients tested showed any interference. Assay results were found to be within the target for all 4 formulations (i.e. Assay NLT 95% and NMT 105%).

### Droplet Size Distribution (DSD)—Method Development

Droplet size measurement of the aerosol emitted from the selected pumps was carried out employing Malvern Spraytec (Malvern Panalytical Ltd, UK) in an open bench configuration. The nasal spray was actuated at 3 and 6 cm from the laser in a carefully defined position with an extraction hood on top to ensure safety of the analyst as well as ensure one single crossing of the laser beam by the spray emitted after actuation. Data collected was analyzed in terms of transmittance, volume diameters of the 10th, 50th and 90th percentile of the Gaussian distribution and width of the droplets distribution obtained (span). A Malvern Mastersizer may also be used to analyze DSD.

Analysis was performed as n=6 for each of the four formulations employing two different analysts (n=3 per analyst) in order to reduce manual actuation variability.

The repeatability of the method was assessed from the standard deviation of three replicate measurements performed by two analysts. The combined variability for both analyst was within the specified limits in in the USP <429>, for all measurements except the Dv, 10 for formulation 4 at 6 cm. Formulation 2 tested at 3 cm for one analyst was also slightly higher than the required criteria, in this instance the combined analysis of both analysts was within the 10% criteria.

All formulations showed comparable droplet size distribution (DSD) 15-70 μm, which showed to be suitable for nasal delivery. Particularly, a median droplet size of 30-40 μm was reported to successfully deposit in the nasal cavity (Sangolkar et al., 2012. Particle size determination of nasal drug delivery system: A review. International Journal of Pharmaceutical Sciences Review and Research 17(1), no. 14, 66-73; Trows et al., Analytical Challenges and Regulatory Requirements for Nasal Drug Products in Europe and the U.S., 2014 Apr. 11; 6(2): 195-219). Bigger droplets (higher than 120 μm) would deposit at the front of the nose, while finer particles may penetrate further into the body (lungs), these last two are deposition patterns which has to be avoided for this formulation development.

### Spray Content Uniformity (SCU)—Method Development

A suitable method for the quantification of the amount of peptide emitted at every spray from the four formulations metered dose pumps coupled with nasal actuator was developed. This method was then employed to determine the SCU of the different formulations manufactured into a suitable collection container. The SCU test was performed at the beginning life stage of the liquid products.

Analysis was performed for n=10 sprays for each liquid formulation. Shot weight was recorded as well.

The Avicel formulations at 0.1 and 1.0 mg/mL, with a target delivered dose of 10.00 and 100.00 mcg, respectively, showed a SCU mean of 10.41 mcg+/−0.13 mcg, % RSD 1 and % of TDD 104; and a mean of 100.08 mcg+/−0.93 mcg, % RSD 1 and % of TDD 100, respectively.

The PEG 400 formulations at 1.0 and 0.1 mg/mL, with a target delivered dose of 100.00 and 10.00 mcg, respectively, showed a SCU mean of 98.83+/−1.31 mcg, % RSD 1 and % of TDD 99; and a mean of 9.67+/−0.14 mcg, % RSD 1 and % of TDD 97, respectively.

All formulations presented consistent delivered dose from the Aptar pump, emitting the target amount of API for both formulation strengths. These results were in line with the acceptance criteria from the USP/EP.

### pH

pH value was measured for the liquid formulations developed in order to determine if it is suitable for nasal products and for the peptide stability. Three measurements were carried out for each liquid formulation. The pH values collected for the final formulations were as expected (target pH 4.5), suitable for nasal delivery (Dhakar et al., 2011, A review on factors affecting the design of nasal drug delivery system, International Journal of Drug Delivery 3(2), 194-208), as shown in the table below.

| pH Results | Average |
|---|---|
| Formulation 1 | 4.51 |
| Formulation 2 | 4.51 |
| Formulation 3 | 4.52 |
| Formulation 4 | 4.49 |

Osmolality. Osmolality was checked in triplicate for each liquid formulation by using a 16M Löser freezing point osmometer (Löser Messtachnik, DE). 25 μL of material was pipetted into the system sample holder for the analysis.

| Osmometry Results (mOsm) | Average |
|---|---|
| Formulation 1 | 31.33 |
| Formulation 2 | 32.67 |
| Formulation 3 | 83.67 |
| Formulation 4 | 80.00 |

Osmolality was lower than the optimal osmolality range for nasal formulations, 290-500 mOsm/kg. These low osmolality values were particularly visible with the Avicel containing formulations. The target of 290 mOsm will be obtainable with the addition of tonicity agents such as mannitol, dextrose, sodium chloride, sorbitol of fructose.

### Viscosity

The viscosity of the formulation was measured using Kinexus Ultra+(Malvern Panalytical, UK). The following data output displayed: viscosity average and standard deviation. Viscosity of the nasal spray has an impact on spray characteristics such as droplet size and spray geometry and the nasal deposition. It was reported that nasal formulations with low viscosity tend to deposit distal to the nares compared to viscous formulations (Guo Y, 2005, The effect of formulation variables and breathing patterns on the site of nasal deposition in an anatomically correct model, Pharm Res. 2005 November; 22(11):1871-8; Sosnowski TR, 2020, Impact of physicochemical properties of nasal spray products on drug deposition and transport in the pediatric nasal cavity model, Int J Pharm. 2020 Jan. 25; 574:118911). Besides, it was found that the higher viscosity is associated with larger droplet size and narrower plume angle, leading to smaller spray area (Kundoor V, 2011, Effect of formulation-and administration-related variables on deposition pattern of nasal spray pumps evaluated using a nasal cast Pharm Res. 2011 August; 28(8):1895-904; Pu Y, 2014, A comparison of the deposition patterns of different nasal spray formulations using a nasal cast, Aerosol Science and Technology, 48:9, 930-938). Three samples for each liquid formulation were analyzed for viscosity.

The power law model was fitted on the viscometry data in order to calculate the flow consistency index, k, and the power law index, n. Tangent analysis was applied to the oscillation amplitude data to calculate the cross-over point from the Linear Viscoelatic region (LVR) to the flow region, which is being used to measure of yield stress. Equilibrium flow curves (shear viscosity vs shear rate) and plots from the oscillation amplitude sweep experiments were prepared.

The power law fit describes the behavior of a non-Newtonian fluid for the formulations comprising Avicel. It was observed that n was less than one and the power law predicts that the effective viscosity would decrease with increasing shear rate (shear thinning profile). This is the typical behavior of formulations comprising Avicel [Rudraraju and Wyandt, 2005, Rheological characterization of Microcrystalline Cellulose/Sodiumcarboxymethyl cellulose hydrogels using a controlled stress rheometer: part I, Int J Pharm. 2005 Mar. 23; 292(1-2):53-61]. In contrast, the formulations comprising PEG400 showed a higher variability in the viscosity collected at low shear rates and a Newtonian behavior at higher shear rate.

| Viscosity | | Standard | Average | Standard |
|---|---|---|---|---|
| Results | Average η | Deviation η | k1 | Deviation k1 |
| Formu- | 0.51 | 0.04 | 0.09 | 0.02 |
| lation 1 | | | | |
| Formu- | 0.59 | 0.01 | 0.07 | 0.00 |
| lation 2 | | | | |
| Formu- | 0.92 | 0.06 | 0.00 | 0.00 |
| lation 3 | | | | |
| Formu- | 0.97 | 0.04 | 0.00 | 0.00 |
| lation 4 | | | | |

### Example 5. Powder Formulations of Octreotide for Nose-to-Brain Delivery

A powder formulation of octreotide was developed for delivery using the Aptar UDSp device, which has a recommended fill weight of 10-80 mg. Octreotide formulations comprising 0.1% octreotide were prepared for a 10-80 mcg delivered dose based on a 10-80 mg dispensed weight, and a formulation comprising 0.5% octreotide for a 50-250 mcg delivered dose based on a 10-50 mg dispensed weight.

Method development for powder formulations. Eight different powder formulations—#M1, #M2, #M3, #M4, #C1, #C2, #C3 and #C4—were manufactured by spray drying, based on the following four octreotide compositions (all w/w):
Octreotide 0.1%, mannitol 95%, hydrogenated lecithin 5%.
Octreotide 0.5%, mannitol 95%, hydrogenated lecithin 5%.
Octreotide 0.1%, calcium carbonate 95%, mannitol 2.5%, L-leucine 2.5%
Octreotide 0.5%, calcium carbonate 95%, mannitol 2.5%, L-leucine 2.5%

Formulation code #s: M, mannitol; C, calcium carbonate; 1 or 2, 0.1% octreotide; 3 or 4; 0.5% octreotide; 1 or 3: inlet temperature 100° C.; 2 or 4, inlet temperature 120° C.

The parameters used for spray drying were a feed solution concentration 2% (w/v; total of octreotide and excipients, in water), the Inlet Temperature was either 100 or 120° C., 100% Aspiration and a Flow rate of 600 L/h. The yield of the mannitol formulation was >70% and the calcium carbonate formulation was >50%. Formulations #C1, #C2, #M1 and #M2 had an octreotide concentration of 0.1%(w/w) and formulations #C3, #C4, #M3 and #M4 were 0.5% (w/w). During the drug loading preparations the 0.1 and 0.5% values of the drug weight were subtracted from the mannitol weight, so in effect values of either 94.5 or 94.95% of mannitol were used in #M formulations and 2.45 or 2.49% of mannitol was used in the #C formulations.

The table below displays some representative details of these formulations such as composition, conditions in which the powders were spray dried, yield, residual water content and particle size distribution (PSD).

For powders M1, M2, M3 and M4 the PSD values are relative to the spray dried powder before agglomeration. Powders M1 to M4 were agglomerated by placing the powder on the top of a stack of two sieves with nominal apertures of 600 mm and 106 mm, respectively (10 cm diameter sieves, Endecotts Ltd., London, UK); the final collector was added to the stack. The sieve stack was closed with the glass cover and vibrated for 5 min on a laboratory sieve shaker (amplitude 3; Analysette 3 Fritz model, Fritsch GMBH, Germany). Agglomerates retained between 600 mm and 106 mm were collected. The non-agglomerated powder in the collector was reprocessed, and the large agglomerates on the first sieve were crushed. The entire process was repeated twice. The yield of the agglomeration process was higher than 90% for all the powders.

After several reanalysis, water residual content for powders C1, C2, C3 and C4 was not detectable by TGA. Dv values represent a sphere of the same volume, and percentile of cumulative distribution. Data on the formulations are provided in the following table.

### Octreotide Powder Formulation Characterization

| | | | | Spray Dryer Conditions | | | | | | |
|---|---|---|---|---|---|---|---|---|---|---|
| | | | Theoretical | Inlet | Feed | Spray Dryer | Residual Water | | | |
| Sample | | Weight | OA Conc. | Temperature | Rate | Yield | Content | Dv (10) | Dv (50) | Dv (90) |
| Name | Composition | (g) | (w/w %) | (° C.) | (ml/min) | (%) | (% w/w) | (μm) | (μm) | (μm) |
| Powder | Mannitol | 5.04 | 0.1 | 100 | 5 | 63.14 | 0.14 ± | 0.53 ± | 2.73 ± | 6.63 ± |
| #M1 | Lecithin | | | | | | 0.01 | 0.01 | 0.08 | 0.08 |
| Powder | Mannitol | 5.08 | 0.1 | 120 | 5 | 69.20 | 0.11 ± | 0.57 ± | 2.37 ± | 5.85 ± |
| #M2 | Lecithin | | | | | | 0.01 | 0.00 | 0.01 | 0.03 |
| Powder | Mannitol | 5.12 | 0.5 | 100 | 5 | 61.22 | 0.23 ± | 0.42 ± | 2.46 ± | 6.38 ± |
| #M3 | Lecithin | | | | | | 0.00 | 0.00 | 0.03 | 0.26 |
| Powder | Mannitol | 4.14 | 0.5 | 120 | 5 | 42.88 | 0.11 ± | 0.68 ± | 2.01 ± | 6.15 ± |
| #M4 | Lecithin | | | | | | 0.01 | 0.01 | 0.01 | 0.15 |
| Powder | CaCO3 | 5.92 | 0.1 | 100 | 5 | 58.84 | 0 | 3.29 ± | 14.40 ± | 26.20 ± |
| #C1 | Mannitol | | | | | | | 1.60 | 0.47 | 0.13 |
| | L-Leucine | | | | | | | | | |
| Powder | CaCO3 | 5.30 | 0.1 | 120 | 5 | 51.25 | 0 | 4.55 ± | 14.50 ± | 26.00 ± |
| #C2 | Mannitol | | | | | | | 0.30 | 0.11 | 0.20 |
| | L-Leucine | | | | | | | | | |
| Powder | CaCO3 | 5.49 | 0.5 | 100 | 5 | 58.68 | 0 | 3.69 ± | 14.20 ± | 25.40 ± |
| #C3 | Mannitol | | | | | | | 1.03 | 0.16 | 0.12 |
| | L-Leucine | | | | | | | | | |
| Powder | CaCO3 | 5.89 | 0.5 | 120 | 5 | 59.66 | 0 | 5.37 ± | 14.10 ± | 25.10 ± |
| #C4 | Mannitol | | | | | | | 0.44 | 0.25 | 0.25 |
| | L-Leucine | | | | | | | | | |

### Example 6. Further Characterization of Powder Formulations of Octreotide for Nose-to-Brain Delivery

Spray Content Uniformity. Calcium carbonate based low dose formulations have an expected content of 0.1% (w/w) and a target delivered dose of 50 mcg. Formulation C1 had a mean SCU of 16.60 (SD 1.10; % RSD 6.65, % of TDD 33.20), and Formulation C2 40.24 (SD 1.76, % RSD 4.38, % of TDD 80.47). Calcium carbonate based high dose formulations have an expected content of 0.5% (w/w) and a target delivered dose of 250 mcg. High dose Formulation C3 had a mean SCU of 196.35 (SD 16.33, % RSD 8.32, % of TDD 78.54), and Formulation C4 has a SCU of 173.96 (SD 17.75, % RSD 10.20, % of TDD 69.58). The recoveries from the single 25 mg shots of either concentration were recovered from 10 mL volumetric, filtered through a nylon 0.2 μm filter into a HPLC vial for testing. The #C1 showed the lowest recovery obtained from all eight formulations at ˜33% of the TTD.

The #C2 formulation had a better recovery at #80% of the TTD, and was more consistent with both the higher doses of #C3 ˜79%, and #C4˜70%.

The data was consistent within batches as displayed by the relatively low % RSD values that ranged from ˜4-10%.

These data sets align with the assay results obtained by HPLC, which indicated that the active was lost in other regions of the spray drier hence the low drug loading.

Mannitol based low dose formulations have an expected content of 0.1% (w/w) and a target delivered dose of 50 mcg. The mannitol-based low dose formulations Formulation M1 has a mean SCU of 50.95 (SD 0.89; % RSD 1.74, % of TDD 101.89), and Formulation M2 51.22 (SD 1.36, % RSD 2.65, % of TDD 102.44). Mannitol based high dose formulations have an expected content of 0.5% (w/w) and a target delivered dose of 250 mcg. High dose Formulation M3 has a mean SCU of 251.43 (SD 5.50, % RSD 2.19, % of TDD 100.57), and Formulation M4 has a SCU of 246.81 (SD 3.74, % RSD 1.52, % of TDD 98.72).

As before, the recoveries from the single 25 mg shots of either concentration were recovered from 10 mL volumetric, filtered through a nylon 0.2 μm filter into a HPLC vial for testing. Unlike the #C formulations, there was less variation between the #M formulations which ranged from 98% ( #M4) to 102% ( #M2) of the TTD.

The data was very consistent within batches, even more so that the #C formulations, as displayed by the relatively low % RSD values that ranged from ˜1.5-2.7%.

### Droplet Size Distribution

The DSD for the calcium carbonate formulations are show below.

| | | | |
|---|---|---|---|
| Analyst 1 (JS) | D10 | D50 | D90 |

Formulation #C1 - Distance 3 cm

| | | | |
|---|---|---|---|
| Average | 11.91 | 19.14 | 30.38 |
| STD | 0.27 | 0.39 | 0.61 |
| RSD % | 2.29 | 2.05 | 2.00 |

| | |
|---|---|
| Span | 0.97 |

Formulation #C2 - Distance 3 cm

| | | | |
|---|---|---|---|
| Average | 13.50 | 21.57 | 34.03 |
| STD | 0.59 | 0.71 | 0.80 |
| RSD % | 4.37 | 3.27 | 2.34 |

| | |
|---|---|
| Span | 0.95 |

Formulation #C3 - Distance 3 cm

| | | | |
|---|---|---|---|
| Average | 11.66 | 18.38 | 28.62 |
| STD | 0.64 | 0.49 | 0.07 |
| RSD % | 5.52 | 2.66 | 0.23 |

| | |
|---|---|
| Span | 0.92 |

Formulation #C4 - Distance 3 cm

| | | | |
|---|---|---|---|
| Average | 12.21 | 18.67 | 28.29 |
| STD | 0.23 | 0.42 | 0.79 |
| RSD % | 1.92 | 2.27 | 2.80 |

| | |
|---|---|
| Span | 0.86 |

In general the results from these formulations produced particle diameters of a good value for a nasal spray with D50 values ranging from ˜18 μm (#C3) to ˜22 μm (#C2) indicating both consistency within each batch and between all #C batches.

### Powder Formulation Method Development

The assay recovery for all powder octreotide formulations is shown in the following table.

| Assay Results | Average | % | Target |
|---|---|---|---|
| (Concentration μg/mL) | conc. | Assay | mass (μg). |
| C1 | 32.20 | 32.20 | 100 |
| C2 | 76.60 | 76.60 | |
| C3 | 423.50 | 84.70 | 500 |
| C4 | 397.51 | 79.50 | |
| M1 | 98.06 | 98.06 | 100 |
| M2 | 96.67 | 96.67 | |
| M3 | 468.53 | 93.71 | 500 |
| M4 | 458.27 | 91.65 | |

The assay results mirror the observations seen in the SCU data for all of the formulations. Both the low and high dose formulations performed well with the mannitol based formulations, with the lower dose showing the highest levels of recovery 97-99%. The calcium carbonate based formulation showed the same pattern as the SCU data with very similar values of recovery as well.

### Example 7. Treatment of Idiopathic Intracranial Hypertension Using Octreotide Nose-to-Brain Liquid Formulation

A patient suffering from idiopathic intracranial hypertension is started on a course of treatment with an intranasal formulation of octreotide acetate delivered using a nose-to-brain delivery as described herein. A liquid formulation comprising octreotide 0.1 mg/mL, Avicel CL611 2% (w/v), polysorbate 80 2% (w/v), BAC 0.02% (w/v) in 0.01M citrate buffer pH 4.5 is used; the tonicity is increased with NaCl to about 290 mOsm/kg. Delivery is using a VP7-232NE device (Aptar Pharma). The device delivers 100 mcl per pump. The dose of octreotide is 10 mcg per pump. The patient is started on a daily dosing regimen, which will be increased to twice or three times daily if clinical effects are not achieved using a single daily dose. Within days from the initiation of daily dosing, the patient experiences relief from headache. Intracranial pressure is also found to be reduced. No side effects are associated with the administration.

### Example 8. Treatment of Cluster Headache Using Octreotide Nose-to-Brain Liquid Formulation

A patient suffering from cluster headaches without relief from any therapy during or between episodes is started on a course of treatment with an intranasal formulation of octreotide acetate delivered using a nose-to-brain delivery device. The formulation is octreotide 1.0 mg/mL, PEG 400 2% (w/v), polysorbate 80 2% (w/v), BAC 0.02% (w/v) in 0.01M citrate buffer pH 4.5, The tonicity is increased with NaCl; delivery is using a VP7-232NE device (Aptar Pharma). The device delivers 100 mcl per pump. The dose is 100 mcg per pump. Treatment is started at the onset of a headache cycle. The patient is started on a daily dosing regimen, which will be increased to twice or three times daily if clinical effects are not achieved using a single daily dose. Within one week from the initiation of daily dosing, the patient experiences a reduction in frequency of headaches which breaks the attack cycle. The patient is maintained on a less frequent dosing schedule which prevents recrudescence.

### Example 9. Treatment of Idiopathic Intracranial Hypertension Using Octreotide Nose-to-Brain Powder Formulation

A patient suffering from idiopathic intracranial hypertension is started on a course of treatment with an intranasal formulation of octreotide acetate delivered using a nose-to-brain delivery formulation and device. The formulation is octreotide 0.1%, mannitol 95%, hydrogenated lecithin 5%. An Aptar UDSp device is used for delivery; each pump delivers 10 mg. The dose is 10 mcg per pump. The patient is started on a daily dosing regimen, which will be increased to twice or three times daily if clinical effects are not achieved using a single daily dose. Within days from the initiation of daily dosing, the patient experiences relief from headache. Intracranial pressure is also found to be reduced. No side effects are associated with the administration.

### Example 10. Treatment of Cluster Headache Using Octreotide Nose-to-Brain Powder Formulation

A patient suffering from cluster headaches without relief from any therapy during or between episodes is started on a course of treatment with an intranasal formulation of octreotide acetate delivered using a nose-to-brain delivery device. The formulation is octreotide 0.5%, mannitol 95%, hydrogenated lecithin 5%. The tonicity is increased with NaCl. An Aptar UDSp device is used for delivery; each pump delivers 50 mg. The dose is 250 mcg per pump. Treatment is started at the onset of a headache cycle. The patient is started on a daily dosing regimen, which will be increased to twice or three times daily if clinical effects are not achieved using a single daily dose. Within one week from the initiation of daily dosing, the patient experiences a reduction in frequency of headaches which breaks the attack cycle. The patient is maintained on a less frequent dosing schedule which prevents recrudescence.

1. A method for treating idiopathic intracranial hypertension (IIH) or cluster headache in a subject in need thereof, the method comprising administering to the subject an effective intranasal amount of a somatostatin mimetic formulated for direct nose-to-brain administration.

2. The method of claim 1, wherein the effective intranasal amount is equal to or less than about 100 mcg, 50 mcg, 10 mcg, 5 mcg or 1 mcg.

3. The method of claim 1, wherein the effective intranasal amount is less than the amount effective when the somatostatin mimetic is administered by a non-intranasal route in accordance with the same dosing regimen.

4. The method of claim 1, wherein a serum area-under-the-curve from a unit dose of the intranasal amount of the somatostatin mimetic is less than the serum area-under-the-curve of an effective unit dose of the somatostatin mimetic administered by a non-intranasal route.

5. The method of claim 1, wherein a serum area-under-the-curve from a unit dose of the intranasal amount of the somatostatin mimetic is less than the serum area-under-the-curve from an effective unit dose of the somatostatin mimetic administered by the intranasal route for an indication other than IIH or cluster headache.

6. The method of claim 5 wherein the indication other than IIH or cluster headache is acromegaly or carcinoid syndrome.

7. The method of claim 3, wherein the non-intranasal route is subcutaneous, intramuscular, oral or intravenous.

8. The method of claim 4, wherein the non-intranasal route is subcutaneous, intramuscular, oral or intravenous.

9. The method of claim 4, wherein the serum area-under-the-curve of the intranasal amount of the somatostatin mimetic is about equal to or less than about 50%, 25%, or 10% of the serum area-under-the-curve of the somatostatin mimetic administered by a non-intranasal route.

10.-11. (canceled)

12. The method of claim 5, wherein the serum area-under-the-curve of the intranasal amount of the somatostatin mimetic is about equal to or less than about 50%, 25% or 10% of the serum area-under-the-curve of the somatostatin mimetic administered by the intranasal route for an indication other than IIH or cluster headache.

13.-14. (canceled)

15. The method of claim 1, wherein the effective intranasal amount is administered as a single daily dose or as 2, 3 or 4 divided doses.

16. (canceled)

17. The method of claim 1, wherein the somatostatin mimetic is selected from somatostatin, octreotide, lanreotide, pasireotide, pentetreotide, or any combination thereof.

18. The method of claim 1, wherein the somatostatin mimetic formulated for intranasal administration comprises a powder, liquid or gel.

19. (canceled)

20. The method of claim 1, wherein treating is to reduce or prevent an acute, ongoing episode, and/or prophylactic and/or maintenance therapy to prevent future episodes.

21. A pharmaceutical composition in unit dose form comprising a somatostatin mimetic formulated for nose-to-brain administration suitable for treatment of idiopathic intracranial hypertension (IIH) or cluster headache.

22. The pharmaceutical composition in unit dose form of claim 21, wherein the unit dose is equal to or less than about 100 mcg, 50 mcg, 10 mcg, 5 mcg or 1 mcg.

23. The pharmaceutical composition of claim 21, wherein the unit dose comprising the somatostatin mimetic contains less somatostatin mimetic than would be effective if administered by a non-intranasal route following the same dosing regimen.

24. The pharmaceutical composition of claim 21, wherein a serum area-under-the-curve of a unit dose of the somatostatin mimetic administered intranasally is less than the serum area-under-the-curve of the unit dose of the somatostatin mimetic administered by a non-intranasal route.

25. The pharmaceutical composition of claim 23, wherein the non-intranasal route is subcutaneous, intramuscular oral or intravenous.

26. The pharmaceutical composition of claim 24, wherein the non-intranasal route is subcutaneous, intramuscular oral or intravenous.

27.-33. (canceled)

34. The pharmaceutical composition of claim 21, wherein a serum area-under-the-curve from a unit dose of the intranasal amount of the somatostatin mimetic is less than the serum area-under-the-curve from an effective single dose of the somatostatin mimetic administered by the intranasal route for an indication other than IIH or cluster headache.

35. The pharmaceutical composition of claim 34, wherein the indication other than IIH or cluster headache is acromegaly or carcinoid syndrome.

36. The pharmaceutical composition of claim 21, wherein the unit dose is labeled for administration in a single daily dose or as 2, 3 or 4 divided doses.

37. The pharmaceutical composition of claim 21, wherein the unit dose is labeled for daily administration for a duration effective to treat or resolve the IIH or cluster headache.

38. The pharmaceutical composition of claim 21, wherein treatment is to reduce or prevent an acute, ongoing episode, and/or prophylactic and/or maintenance therapy to prevent future episodes.

39. The pharmaceutical composition of claim 21, wherein the somatostatin mimetic is selected from somatostatin, octreotide, lanreotide, pasireotide, pentetreotide, or any combination thereof.

40. The pharmaceutical composition of claim 21, wherein the somatostatin mimetic formulated for nose-to-brain administration comprises a powder, liquid or gel.

41. (canceled)

42. A liquid pharmaceutical composition comprising:
octreotide 0.1 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5;
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

43.-53. (canceled)
